SEQUENCE LISTING (28 sequences)

SEQ ID NO: 1 - protein, 20 aa, Artificial sequence
  A synthetic HCV Core P1 amino acid sequence
MSTNPKPQRKTKRNTNRRPQ

SEQ ID NO: 2 - protein, 20 aa, Artificial Sequence
  A synthetic HCV core P2 amino acid sequence
TKRNTNRRPQDVKFPGGGQI

SEQ ID NO: 3 - protein, 20 aa, Artificial sequence
  A synthetic HCV core P3 amino acid sequence
DVKFPGGGQIVGGVYLLPRR

SEQ ID NO: 4 - protein, 20 aa, Artificial sequence
  A synthetic HCV core P4 amino acid sequence
VGGVYLLPRRGPRLGVRATR

SEQ ID NO: 5 - protein, 20 aa, Artificial sequence
  A synthetic HCV core P5 amino acid sequence
GPRLGVRATRKTSERSQPRG

SEQ ID NO: 6 - protein, 20 aa, Artificial sequence
  A synthetic HCV core P6 amino acid sequence
KTSERSQPRGRRQPIPKDRR

SEQ ID NO: 7 - protein, 20 aa, Artificial sequence
  A synthetic HCV core p7 amino acid sequence
RRQPIPKDRRSTGKSWGKPG

SEQ ID NO: 8 - protein, 20 aa, Artificial Sequence
  A synthetic HCV core P8 amino acid sequence
STGKSWGKPGYPWPLYGNEG

SEQ ID NO: 9 - protein, 20 aa, Artificial sequence
  A synthetic HCV core P9 amino acid sequence
YPWPLYGNEGLGWAGWLLSP

SEQ ID NO: 10 - protein, 20 aa, Artificial sequence
  A synthetic HCV core P10 amino acid sequence
LGWAGWLLSPRGSRPSWGPN

SEQ ID NO: 11 - protein, 20 aa, Artificial sequence
  A synthetic HCV core P11 amino acid sequence
RGSRPSWGPNDPRHRSRNVG

SEQ ID NO: 12 - protein, 20 aa, Artificial sequence
  A synthetic HCV core P12 amino acid sequence
DPRHRSRNVGKVIDTLTCGF

SEQ ID NO: 13 - protein, 20 aa, Artificial sequence
  A synthetic HCV core P13 amino acid sequence
KVIDTLTCGFADLMGYIPVV

SEQ ID NO: 14 - protein, 20 aa, Artificial sequence
  A synthetic HCV core P14 amino acid sequence
ADLMGYIPVVGAPLGGVARA

SEQ ID NO: 15 - protein, 20 aa, Artificial sequence
  A synthetic HCV core P15 amino acid sequence
GAPLGGVARALAHGVRVLED

SEQ ID NO: 16 - protein, 20 aa, Artificial sequence
  A synthetic HCV core P16 amino acid sequence
LAHGVRVLEDGVNFATGNLP

SEQ ID NO: 17 - DNA, 24 bp, Artificial sequence
  A synthetic human GAPDH Forward primer (5′-3′)
gcttaactctggtaaagtggatat

SEQ ID NO: 18 - DNA, 23 bp, Artificial sequence
  A synthetic human GAPDH Reverse primer (5′-3′)
atggaatttgccatgggtggaat

SEQ ID NO: 19 - DNA, 23 bp, Artificial sequence
  A synthetic mouse GAPDH Forward primer (5′-3′)
catttgcagtggcaaagtggaga

SEQ ID NO: 20 - DNA, 23 bp, Artificial sequence
  A synthetic mouse GAPDH Reverse primer (5′-3′)
gttgaatttgccgtgagtggagt

SEQ ID NO: 21 - DNA, 22 bp, Artificial sequence
  A synthetic human Col1A1 Forward primer (5′-3′)
ccaatcacctgcgtacagaacg

SEQ ID NO: 22 - DNA, 22 bp, Artificial sequence
  A synthetic human Col1A1 Reverse primer (5′-3′)
cgtcacagatcacgtcatcgca

SEQ ID NO: 23 - DNA, 21 bp, Artificial sequence
  A synthetic mouse Col1A1 Forward primer (5′-3′)
agctttgtggacctccggctc

SEQ ID NO: 24 - DNA, 23 bp, Artificial sequence
  A synthetic mouse Col1A1 Reverse primer (5′-3′)
ctgacttcagggatgtcttcttg

SEQ ID NO: 25 - DNA, 20 bp, Artificial sequence
  A synthetic human TIMP1 Forward primer (5′-3′)
cccacaaccgcagcgaggag

SEQ ID NO: 26 - DNA, 20 bp, Artificial sequence
  A synthetic human TIMP1 Reverse primer (5′-3′)
ggcaggcaaggtgacgggac

SEQ ID NO: 27 - DNA, 22 bp, Artificial sequence
  A synthetic mouse TIMP1 Forward primer (5′-3′)
ctggcatctggcatcctcttgt

SEQ ID NO: 28 - DNA, 22 bp, Artificial sequence
  A synthetic mouse TIMP1 Reverse primer (5′-3′)
agttgcagaaggctgtctgtgg

(19) United States
(12) Patent Application Publication
(10) Pub. No.: US 2016/0135437 A1
(43) Pub. Date: May 19, 2016

(54) HUMANIZED MOUSE MODEL FOR STUDY OF BONA FIDE HEPATITIS VIRUS INFECTION AND USE THEREOF

(71) Applicant: Agency for Science, Technology and Research, Singapore (SG)

(72) Inventors: Qingfeng CHEN, Proteos (SG); Choong Tat KENG, Proteos (SG); Yee Joo TAN, Proteos (SG); Antonio BERTOLETTI, Proteos (SG)

(21) Appl. No.: 14/896,239

(22) PCT Filed: Jun. 5, 2014
(86) PCT No.: PCT/SG2014/000259

§ 371 Date: Dec. 4, 2015

(30) Foreign Application Priority Data
Jun. 5, 2013 (SG) 201304370-8

(51) Int. Cl. A01K 67/027; A61K 49/00

(52) U.S. Cl. CPC A01K 67/0271; A61K 49/0008; A01K 2217/054; A01K 2227/105; A01K 2267/0337; A01K 2207/12

(57) ABSTRACT

This invention refers to a mouse for human hepatitis studies wherein the mouse has been injected with CD34+ stem cells and wherein the mouse is immunocompromised, as well as a method of manufacturing a mouse model comprising administering CD34+ stem cells as defined herein into an immunocompromised mouse as defined herein. The invention also refers to the use of a mouse as defined for testing the efficiency of putative anti-HBV or anti-HCV drugs or for characterizing changes in viral quasispecies during HBV or HCV infection.

### CROSS-REFERENCE TO RELATED APPLICATIONS

This application claims the benefit of priority of Singapore Patent application No. 201304370-8, filed Jun. 5, 2013, the contents of it being hereby incorporated by reference in its entirety for all purposes.

### FIELD OF THE INVENTION

The present invention relates generally to the field of biotechnology. In particular, the present invention relates to the generation of humanized mice as models for studying human hepatitis virus infection.

### BACKGROUND OF THE INVENTION

Hepatitis viruses, particularly hepatitis B virus (HBV) and hepatitis C virus (HCV) infections, represent a major health concern worldwide. About a third of the world population has been infected with HBV or HCV at one point in their lives and many of them developed severe liver diseases and cancers. There are approximately 300 million chronic HBV carriers in the world, of which 75% are found in the Asia Pacific region. Hepatitis B virus (HBV) is the most common human hepatitis virus in Singapore. Around 6% of the Singaporean population is carrying HBV. Hepatitis C (HCV) is the most common chronic blood-borne infection in the United States. Currently, there are no effective vaccines or antiviral treatments available for HCV or HBV infections, or related virus-associated cancers, making HCV and HBV an economical and public health threat in the world. A major obstacle in the development of vaccine and antiviral therapy is the fact that HBV and HCV tropism is restricted to humans.

The in vivo study of pathology and immune responses against HCV and HBV has been greatly hampered by the lack of a robust, small animal model that can recapitulate HCV virus infection, its immunopathogenesis and disease progression, resulting in the impediment in the development of an effective vaccine and other therapeutics. Furthermore, this lack of appropriate models that accurately replicate human disease and, physiology has no doubt contributed to the high failure rate (50%) of investigational new drugs at phase II clinical trials, along with a lack of efficacy. Although chimpanzees have played a critical role in studying HCV infection, there are several drawbacks, including low chronic infection rate and lack of liver fibrosis, as well as costs and ethical concerns, that limit their utility. A mouse engrafted with human liver cells and a functional human immune system is an excellent model for studying the virus. A number of human-mouse chimeric liver models have been developed, but allow analyses of only limited aspects of HCV infection and pathogenesis. Hence, there is a need for an animal model that is capable of supporting hepatitis infection, in order to further investigate the hepatitis virus.

### SUMMARY

In a first aspect, the present invention refers to a mouse or mouse model for human hepatitis studies wherein the mouse has been injected with CD34+ stem cells and wherein the mouse is immunocompromised.

In a second aspect, the present invention refers to a mouse for human hepatitis studies obtained by a process of injecting CD34+ stem cells into an immunocompromised mouse.

In a third aspect, the present invention refers, to a method of manufacturing a mouse model comprising administering CD34+ stem cells as defined herein into an immunocompromised mouse as defined herein.

In a fourth aspect, the present invention refers to a method of drug screening wherein the method comprises administering anti-HBV or anti-HCV therapeutics to a mouse as defined herein.

In a fifth aspect, the present invention refers to a method of characterizing changes in viral quasispecies during HBV or HCV infection by using a mouse as defined herein.

In a sixth aspect, the present invention refers to use of a mouse as defined herein for testing the efficiency of putative anti-HBV or anti-HCV drugs or for characterizing changes in viral quasispecies during HBV or HCV infection.

### BRIEF DESCRIPTION OF THE DRAWINGS

The invention will be better understood with reference to the detailed description when considered in conjunction with the non-limiting examples and the accompanying drawings, in which:

FIG. 1 shows that HCV can infect mice, as described herein, leading to liver inflammation and liver injury. (A) Ten-week old mice were mock-infected (top row) or HCV-infected (bottom row) for 5 weeks (n=5 mice per group). Livers were harvested and paraffin sections were prepared. Representative stains for DAPI (blue), anti-HCV core (green) and anti-human albumin (red) is shown. (B-C) Ten-week old mice were infected with HCV for 0, 1, 3, 5 and 9 weeks (n=5 mice per group). Livers and sera were harvested for histology and ALT assay. (B) Representative liver H&E stains with lower (top row) and higher (bottom row) magnification from different time points are shown. (C) Serum ALT levels in mice from different time points were represented. Data represents mean±standard error of mean (SEM).

FIG. 2 shows images illustrating that HCV-infected mice, as described herein, develop liver fibrosis. (A-B) Ten-week old mice were infected with HCV for 0, 1, 3, 5 and 9 weeks (n=10 mice per group). Livers were harvested and paraffin sections were prepared. (A) Representative stains for Fast green and Sirius Red from different time points are shown. (B) Representative stains of liver sections from mice, as described herein, that were mock-infected or 9 weeks HCV-infected for alpha smooth muscle actin (αSMA) (green) and DAPI (blue) are shown. (C) Total liver RNAs were prepared from mock-infected and 9 weeks HCV-infected mice (n=3 mice per group). Relative gene expression levels of human TIMP1 and Col1A1 (left), and mouse TIMP1 and Col1A1 (right) were represented in the graphs. Data represents mean±SEM.

FIG. 3 shows that HCV infection in mice, as described herein, leads to intrahepatic human T-cell and macrophage infiltration and cytokine responses. (A) Livers sections were prepared from mice, as described herein, that were mock-infected or HCV-infected for 5 weeks. Representative stains for human CD45 (red) and DAPI (blue) are shown (n=5 mice per group). (B-D) Total intrahepatic mononuclear cells (MNCs) were isolated from mice, as described herein, that were infected for 0, 1, 3, 5, 7 and 9 weeks (n=5 mice per group). Data represents mean±SEM. (B) The numbers of human CD45+ cells were determined from the percentages of CD45+ cells among total hepatic MNCs and the absolute cell number count. (C) The numbers of human CD45+CD3+ T-cells were determined from the percentages of CD45+CD3+ cells among total hepatic MNCs and the absolute cell number count. (D) The number of CD45+CD14+ cells was determined from the percentage of CD45+CD14+ macrophages among total hepatic MNCs and the absolute cell number count. (E-F) Sera were prepared from mice, as described herein, that were infected for 0, 1, 3, 5, 7 and 9 weeks and analysed for human IFN-γ and IL-6 by ELISA (n=5 mice per group). Data represents mean±SEM. (E) Serum levels of human IFN-γ. (F) Serum levels of human IL-6.

FIG. 4 shows a micrograph and a column graph, indicating that HCV infection induces HCV-specific human T-cell response in mice, as described herein. Mice as described herein were mock-infected or HCV-infected for 9 weeks (n=3 mice per group). Spleen mononuclear cells (MNCs) were isolated and 1×10⁵ MNCs were used for stimulation with 16 20-mer HCV core peptides for 2 days for human IFN-γ ELISPOT assay. (A) Representative ELISPOT images of mock-stimulated (Ctrl) and peptide-stimulated MNCs from mock- and HCV-infected mice are shown. PMA-ionomycin stimulated cells were used as positive control. (B) HCV peptide specific human IFN-γ T-cell responses from mock- and HCV-infected mice are shown as spots forming units (SFU) per 106 MNCs. Data represents mean±SEM.

FIG. 5 shows images illustrating that depletion of CD3+ T-cells or CD14+ macrophages in mice, as described herein, significantly reduces inflammation, fibrosis and immune responses caused by HCV infection. Anti-human CD4, CD8 and CD14 antibodies were used to deplete human T-cells and macrophage in mice, as described herein. Livers and sera were harvested from mice 8 weeks after HCV infection (n=5 mice per group). (A) Representative images of livers from PBS-treated control (Ctrl), CD4+CD8 antibodies treated and CD14 antibody treated mice (top row) were shown. Representative liver stains of H&E to visualize leukocyte infiltration (middle row) and stains for Fast Green & Sirius Red to visualize collagen deposition (bottom row) are shown. (B) Serum levels of human IFN-γ (left) and human IL-6 (right) were determined by ELISA. Data represents mean±SEM.

FIG. 6 shows micrograph and stained liver sections illustrating that chronic HCV infection of mice, as described herein, induces hepatoma formation. Livers and sera were harvested from mock-infected or 27-weeks HCV-infected mice (n=3 mice per group). (A) Representative image of liver tumors from 27-weeks HCV-infected mice. (B) Representative H&E stains of liver tumors. (C) Representative H&E stains of necrosis (black arrow) and hepatocyte mitosis (white arrow) in liver tumors. (D) Representative stains for human albumin (white area) of liver tumors. (E) Serum levels of human albumin were determined by ELISA. Data represents mean±SEM.

FIG. 7 shows micrograph illustration that clinical strain HCV infection results in liver leukocyte infiltration and fibrosis in mice, as described herein. Livers sections were prepared from mice that were mock-infected and 13-week clinical strain (CS) HCV-infected (n=5 mice per group). (A) Representative H&E (top row) and Fast Green & Sirius Red (bottom row) stains are shown. (B) Representative stains for human CD45 (first column) and DAPI (second column) are shown.

FIG. 8 shows scatter plot data, as well as micrographs, illustrating that NSG mice support the engraftment of human hepatocytes and a matching human immune system. New born NSG mice were injected with 2×10⁵ fetal liver CD34+ cells to construct mice, as described herein. Eight weeks after injection, PBMCs, sera and liver paraffin sections were prepared from NSG and mice, as described herein, (n=50 mice per group). (A) PBMCs were stained for human CD45 (hCD45) versus mouse CD45.1 (mCD45) to determine the reconstitution level of human leukocytes [% human CD45+ cells/(% human CD45+ cells+% mouse CD45+ cells)]. Shown are representative plots of hCD45 versus mCD45.1. (B) The reconstitution level of human leukocytes in individual NSG and mice as described herein were tabulated and represented in the graph. Each symbol represents one mouse. (C) Liver sections from NSG and mice as described herein were stained for human albumin (first column) and DAPI (second column). Representative stains are shown. (D) Serum levels of human albumin in NSG and mice as described herein were tabulated and represented in the graph. Each symbol represents one mouse. Data represents mean.

FIG. 9 shows micrograph images of the Haematoxylin & Eosin staining (H&E) of livers of Balb/c, NSG and cord blood (CB) reconstituted humanized mice, which had been infected with HCV for 0, 1, 3, 5, 7 and 9 weeks (n=5 mice per group). Livers were harvested from different time points and paraffin sections were prepared. Representative H&E stains of liver sections from the week-9 time point are shown.

FIG. 10 shows the images of livers from mice, as described herein, which were mock-infected or HCV-infected for 9 weeks (n=10 mice per group). Representative liver images are shown. Hepatoma formation is visible on the HCV infected mouse liver on the right of the image.

FIG. 11 shows graphs depicting the levels of human IFN-γ and IL-6 in cord-blood reconstitute (CB) mice, which were infected with HCV for 0, 1, 3, 5, 7 and 9 weeks (n=5 mice per group). Sera were prepared from different time points and analysed for human IFN-γ and IL-6 by ELISA. Data represents mean±SEM. (A) Serum levels of human IFN-γ are shown. (B) Serum levels of human IL-6 are shown.

FIG. 12 shows scatter plots illustration the depletion effects of human T-cells and macrophages in mice as described herein. Mice, as described herein, were treated with PBS (Ctrl), anti-human CD4 and CD8 antibodies or anti-human CD14 antibody (n=3 mice per group). Mononuclear cells (MNCs) were prepared from bloods, spleens and livers 24 h after antibody treatments. (A) MNCs from different organs were stained for human CD3. Shown are representative plots of Forward-scattered light (FSC) versus CD3. (B) MNCs from different organs were stained for human macrophage mannose receptor (MMR). Shown are representative plots of Forward-scattered light (FSC) versus MMR.

FIG. 13 shows graphs depicting the levels of human IFN-γ and IL-6 in HCV clinical strain (genotype 3a)-infected mice, which were infected for 0, 1, 3, 5, 7 and 9 weeks (n=5 mice per group). Sera were prepared from different time points and analysed for human IFN-γ and IL-6 by ELISA. Data represents mean±SEM. (A) Serum levels of human IFN-γ are shown. (B) Serum levels of human IL-6 are shown.

FIG. 14 illustrates the detection of HBV viremia in HBV infected mice as described herein. (A) shows viremia in the serum of HBV-infected mice as described herein. The viremia was detected in the serum of some HBV-infected mice was detected at a level of 102 IU/ml. (B) shows viremia present in the livers of HBV-infected mice. For mice with no detectable viremia in the serum, viremia was detected in the livers, with the levels ranging from 102 to 103 IU/μg RNA.

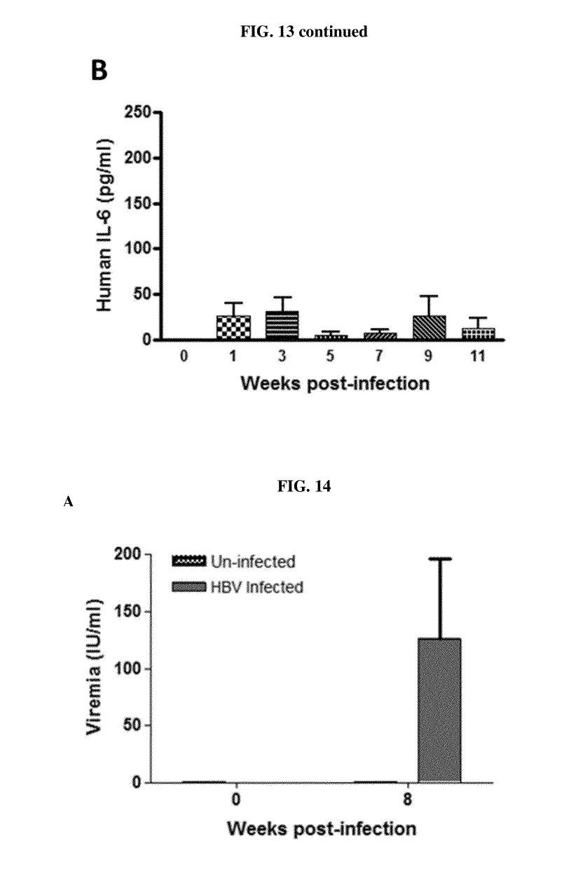

FIG. 15 shows fluorescent micrographs illustrating the hepatitis B virus (HBV) Pre-S2 antigen staining in hepatocytes of HBV-infected mice, as described herein, illustrating that the HBV HBsAg antigen can be detected by immunostaining in the livers of the infected mouse as early as 8 weeks post-infection, thus proving the susceptibility of mice as described herein to the infections agent HBV.

FIG. 16 shows data used to illustrate the human cytokine responses elicited in HBV-infected mice, as described herein. (A-B) show that human interferon-gamma levels in the serum of HBV-infected mice were detected at high levels, with the concentration peaking at 8-14 weeks post-infection. (C-D) show human IL-6 levels in the serum of HBV-infected mice as described herein were detected at high levels, with the concentration also peaking at 8-14 weeks post-infection.

FIG. 17 depicts micrographs showing liver immune-cell infiltration in hepatocytes of HBV-infected mice as described herein. H&E staining of the cells shows a massive immune cell infiltration in the livers of the HBV-infected mouse as early as 8 weeks post-infection.

FIG. 18 depicts micrographs showing liver fibrosis immunohistochemically stained liver sections of HBV-infected mice as described herein. Sirius red and Fast green staining revealed liver fibrosis in the livers of HBV-infected mouse as early as 8 weeks post-infection.

FIG. 19 shows fluorescent microscopy imaged, illustrating the presence of HBV-specific human antibodies in HBV infected humanized mice, as described herein. The negative control was uninfected mouse serum. Detection of anti-human IgG-FITC shows the co-localization of anti-human IgG at the periphery of infected cells.

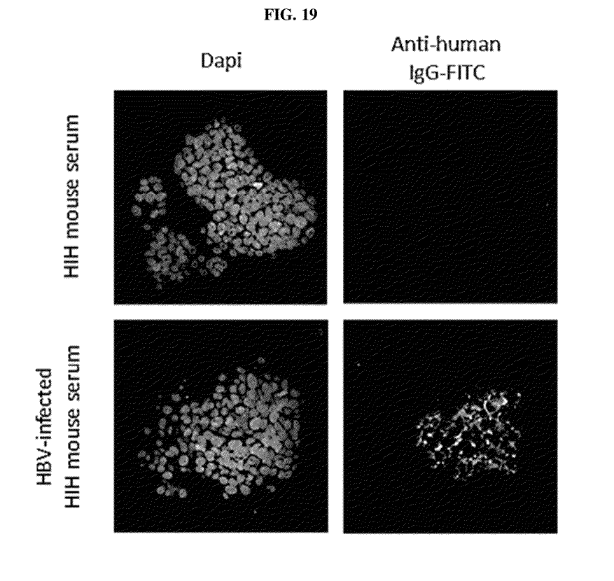

### DETAILED DESCRIPTION OF THE PRESENT INVENTION

Hepatitis viruses, particularly hepatitis C virus (HCV) represents a major health concern worldwide, with more than 175 million people infected. Many of these patients with the infection often progress to develop hepatitis, liver fibrosis, cirrhosis and hepatocellular adenoma or carcinoma. Currently, there is no vaccine known to be effective and limited options are available for antiviral treatments, making HCV an economic and public health threat to the world. A major obstacle in the development of vaccine and antiviral therapy arises from the fact that HCV tropism is restricted to humans, thus resulting in little to no infectivity of the virus in other hosts. Chimpanzees are currently the most complete model able to support the complete HCV life cycle and recapitulate the host response observed in human patients, but limitations such as low chronic infection rate, poor demonstration of liver fibrosis, high cost and ethical concerns have limited their usage for HCV research.

To add to the challenge, HCV is not able to replicate in cell culture and as a result, research using different clinical HCV strains is unable to achieve consistency and continuity, which is also compounded by limited availability of the amount of a particular clinical HCV strain used in testing. The efficient replication of HCV was shown to be achievable by cell culture adaptation, but mutation lead to the inhibition of virion assembly and attenuate RNA infectivity in vivo. A genotype 2a HCV strain, JFH-1, was identified in the art to have efficient RNA replication in cell culture without the need for adaptive mutations. This strain was further improved by replacing the region from core to non-structural protein 2 with the same region of the HCV strain J6, increasing production of infectious virus (FL-J6/JFH) in cell culture (HCVcc). The HCV FL-J6/JFH strain was further shown in the art to be infectious in vivo in chimpanzees and humanized chimeric urokinase-type plasminogen activator (uPA)-severe combined immunodeficiency (SCID) mouse model.

The present description provides a method of manufacturing a mouse model, comprising administering CD34+ stem cells as defined herein into an immunocompromised mouse as defined herein. In one example, the method is as described herein, wherein said method further comprises administering HBV or HCV into the mouse.

As used herein, the term “CD34+” refers to the presence of the CD34 cluster of differentiation molecule present on certain cells in the human body, as well as the human gene that encodes said protein molecule. The prefix “CD” stands for “cluster of differentiation”. The term “+” denotes the presence of the cluster, whereas “−” denotes the absence thereof. It is a cell surface glycoprotein and functions as a cell-cell adhesion factor. It may also mediate the attachment of stem cells to bone marrow extracellular matrix or directly to stromal cells.

The lack of a small animal model that can recapitulate the viral infection and liver pathogenesis observed in human patients has limited the progress in understanding virus-host interaction, HCV-specific immune responses and progression of the diseased pathology, as well as in the development of vaccines and therapeutics. Early mouse models developed for HCV infection were transgenic mouse models that were genetically modified to allow virus infection of murine hepatocytes, or to enhance the transplantation efficacy of mature human hepatocytes. The immunodeficient Alb-uPA/SCID mouse model, of which the liver had been repopulated with mature human hepatocytes, was used to demonstrate successful HCV infection in vivo; however the mouse model is known to be not without issues, such as excessive mortality, poor breeding and transgene reversion. The Alb-uPA/SCID transgenic mouse further contains the uPA transgene under control of an albumin promoter. Homozygous animals of this uPA transgene are unhealthy and die due to profound hypofibrinogenemia and accelerated hepatocyte death. These animals, however, can be rescued by transplantation of murine or human hepatocytes. Furthermore, an Alb-uPA/SCID mouse with efficient human hepatocyte engraftment can be infected with HCV.

As used herein, the term “mature” refers to a characteristic that is fully developed.

Another mouse model, the Fah-Rag2-γC null mouse, was later developed to solve most of the limitations associated with the Alb-uPA/SCID mouse, but the Fah-Rag2-γC null mice still lack a functional human immune system for studying HCV immunopathogenesis. It was recently reported that a Fah-Rag2-γC-null mouse can also be highly engrafted with human hepatocytes to support HCV infection. However, due to lack of a functional immune system, it is not possible to study HCV immunopathogenesis and no liver diseases are observed in either the Alb-uPA/SCID or Fah-Rag2-γC-null model.

As of late, the AFC8-HSC/Hep Balb/C Rag2-γC-null mouse model was developed with both a human immune system and human liver cells. This mouse model was shown to be able to support HCV infection with liver inflammation, hepatitis and fibrosis. It was recently shown that transgenes of Caspase 8 fused with FK506 binding domain (FKBP) in Balb/C Rag2-γC-null mouse, allowing the development of both human immune system and liver cells via co-transplantation of human hepatocyte progenitor cells and CD34+ hematopoietic stem cells (HSCs) into the mouse. However, the transplantation of human liver progenitor cells into this transgenic mouse model constitutes a genetic modification and requires extra drug treatment steps to drive the expression of Caspase 8 to induce murine hepatocyte apoptosis, in order to facilitate the engraftment of the human hepatocytes to the mouse liver after human immune cell reconstitution. The advantage to be had is that this mouse model allows for a HCV infection and the study of human immune responses. The disadvantage is that the transgene itself is also known to cause liver damage and elevation of Alanine transaminase (ALT) levels, which interferes the study of virus infection and subsequent drug evaluation. Moreover, the Balb/C Rag2-γC-null mice used have been shown to result in a much weaker human immune cell reconstitution and function than the NOD-SCID IL2Rγ−/− (NSG) mouse, explaining why the former model produces less severe fibrosis and cirrhosis compared to the latter.

As used herein, the term “progenitor” refers to a biological cell that, like a stem cell, has a tendency to differentiate into a specific type of cell, but is already more specific than a stem cell and is pushed to differentiate into its “target” cell. The most important difference between stem cells and progenitor cells is that stem cells can replicate indefinitely, whereas progenitor cells can divide only a limited number of times. Controversy about the exact definition remains and the concept is still evolving. The terms “progenitor cell” and “stem cell” are sometimes equated. Most progenitors are described as oligopotent, and it is in this point of view that they may be compared to adult stem cells.

These mouse models can be efficiently infected with HCV but do not develop the clinical symptoms observed in HCV patients who had robust CD4 and CD8 cytotoxic T-cell responses. However, the immune system of a majority of HCV patients failed to clear the infection and, as a result, developed a chronic infection, which is associated in the art with impaired CD4 and CD8 T-cell functions. This chronic inflammation and impaired T-cell responses in the liver are suggested to contribute to the development of the liver disease. Thus, the introduced mouse models lack a human immune system and as a result cannot be used to study the HCV-specific immune responses. Moreover, key components of HCV pathology liver fibrosis and cirrhosis are absent in these models.

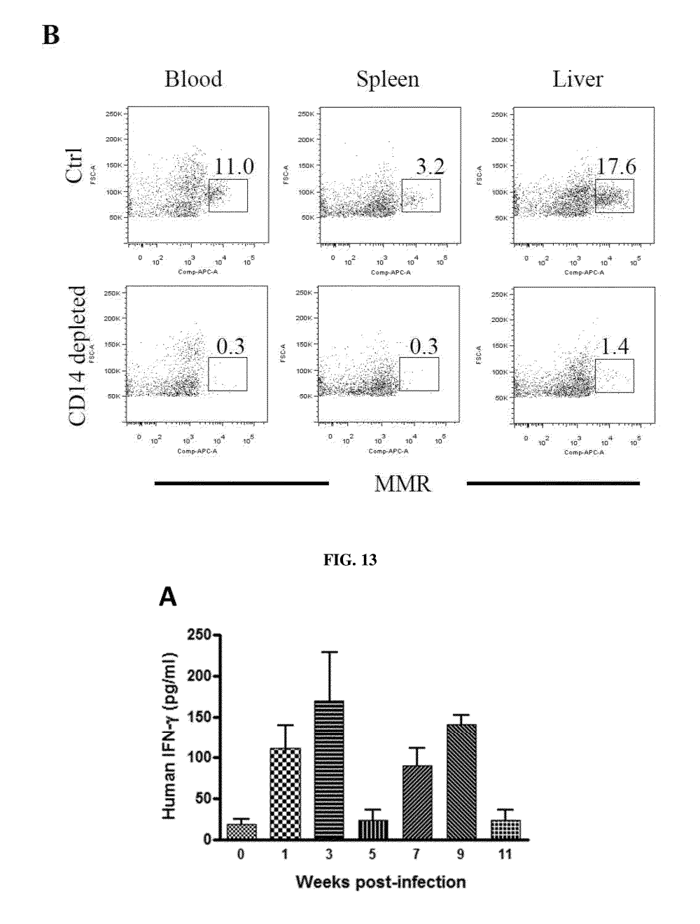

Humanized mice refer to mice that are stably engrafted with human cells or tissues. The development of humanized mice as described in the present disclosure is done by adoptive transfer of human stem cells into NOD scid gamma (NSG) mice, which are utilized as their genetic background is known in the art to be a better recipient for human cell engraftment than Balb/C Rag2-γC-null and display better function reconstitution of human immune cells due to its defective murine phagocyte activity. Recently, using a single-step intrahepatic injection of CD34+ stem cells purified from fresh human fetal liver, it was possible to engraft both the human immune cells (CD45+) and human hepatocytes (Alb+) into the same mouse (FIG. 13). In one example, the mouse is as described herein, wherein the CD34+ stem cells have been injected by one-step injection. In another example, the injection is an intra-hepatic or intra-cardiac injection.

As used herein, the term “CD45” refers to protein tyrosine phosphatase, receptor type, C, which is also known as PTPRC or simply PTPs. PTPs are known to be signalling molecules that regulate a variety of cellular processes including cell growth, differentiation, mitotic cycle, and oncogenic transformation. The term “+” denotes the presence of the cluster, whereas “−” denotes the absence thereof. It is a type I trans-membrane protein that is, in various forms, present on all differentiated hematopoietic cells, with the exception of erythrocytes and plasma cells that assists in the activation of those cells (e.g. as a form of co-stimulation). It is expressed in lymphomas, B-cell chronic lymphocytic leukemia, hairy cell leukemia, and acute non-lymphocytic leukemia. A monoclonal antibody to CD45 is used in routine immunohistochemistry to differentiate between histological sections from lymphomas and carcinomas.

Significant improvements have been made resulting in the currently disclosed mouse model, which will greatly enhance elucidation of cellular and molecular basis of host-pathogen interactions. Most recently, a hepatic stem cell population in human fetal liver has been identified, which leads a simpler method in the construction of a new humanized mouse model with both human hepatocytes in mouse liver and a fully-matched human immune system, effectively creating a humanized mouse model for the study of HCV infection, using a simple, one-step engraftment of human liver cells and a matching human immune system in the same mouse, without the need for transgenic modification or drug treatment.

As used herein, the term “fetal tissue” refers to tissue that is isolated from a fetus. The term “fetal” pertains to, or is connected with, a fetus. In that light, the term “fetus” is defined herein to refer to a developing mammal or other vertebrate after the embryonic stage and before birth. It is also defined as the unborn young of a vertebrate, after developing to its basic form. In humans, the fetal stage of prenatal development may be defined as beginning at the 11^{th} week in gestational age, which is the 9^{th} week after fertilization. In biological terms, however, prenatal development is a continuum, with no clear defining feature distinguishing an embryo from a fetus. The use of the term “fetus” generally implies that a mammalian embryo has developed to the point of being recognizable as belonging to its own species, and this is usually taken to be the 9th week after fertilization. A fetus may also be characterized by the presence of all the major body organs, though they will not yet be fully developed and functional, and may not all be situated in their final anatomical location.

As a result, it has been shown that NSG mice can efficiently develop a functional human immune system and considerable number of human hepatocytes in vivo, thereby generating a mouse model containing both a human immune system and human liver cells (HIL mice). In this disclosure, it is shown that the mice, as described herein, can support HCV infection in the liver, generate robust HCV-specific human immune responses, and develop significant liver diseases, including fibrosis, cirrhosis and hepatitis-associated human hepatomas.

The disclosure describes a mouse for human hepatitis studies, wherein the mouse has been injected with CD34+ stem cells and wherein the mouse is immunocompromised. In one example, the present description provides an immunocompromised mouse, wherein the immunocompromised mouse is characterized by absence of mature T- or B-cells and lack of functional natural killer (NK) cells. In another example, an immunocompromised mouse is further characterized by a deficiency in cytokine signalling. In a further example, there is provided a mouse as described herein, wherein the immunocompromised mouse is further characterized by absence of production of detectable serum immunoglobulin. In another example, the mouse is as described herein, wherein the mouse is a NOD scid IL2 receptor gamma chain knockout mouse (NSG) (NOD-SCID-IL2Rγ−/−).

As used herein, the term “deficiency” refers to an inadequacy or incompleteness. For example, the term “deficiency in cytokine signalling” refers to the fact that a cytokine signalling cascade is incomplete.

As used herein, the terms “immunocompromised” or “immunodeficient” are defined as a state in which the ability of a subject's immune system's to fight infectious disease is compromised or entirely absent. There are different types of immunodeficiency, each depending on which part of the immune system is compromised. One cause may be humoral immune deficiencies, which may or may not be accompanied by cause-dependent indications or symptoms, but generally include signs of hypogammaglobulinemia (decrease of one or more types of antibodies), with presentations including repeated mild respiratory infections, and/or agammaglobulinemia (lack of all or most antibody production). A further cause may be T-cell deficiency, which as the name suggests results from a lack of T-cells, often causing secondary disorders such as acquired immune deficiency syndrome (AIDS). Another cause of an immunodeficiency is granulocyte deficiency, which is defined as a decreased number of granulocytes (i.e. granulocytopenia or, if absent, agranulocytosis), such as neutrophil granulocytes (i.e. neutropenia). Granulocyte deficiencies also include decreased function of individual granulocytes, such as in chronic granulomatous disease. Another possible cause may be asplenia, a condition where there is no function of the spleen, and complement deficiency, where the function of the complement system is deficient.

As used herein, the term “immunoglobulin”, also known as “antibody” refers to an intact antibody, or a binding fragment thereof that competes with the intact antibody for specific binding. Binding fragments are produced by recombinant DNA techniques, or by enzymatic or chemical cleavage of intact antibodies. Binding fragments include Fab, Fab′, F(ab′)2, Fv, and single-chain antibodies. An antibody other than a “bispecific” or “bifunctional” antibody is understood to have each of its binding sites identical. An antibody substantially inhibits adhesion of a receptor to a counter receptor when an excess of antibody reduces the quantity of receptor bound to counter receptor.

As used herein, the term “T-cell” or “T-lymphocytes” refer to a type of lymphocyte that plays a central role in cell-mediated immunity. A T-cell in itself is a type of white blood cell. They can be distinguished from other lymphocytes, such as B-cells and natural killer cells (NK cells), by the presence of a T-cell receptor (TCR) on the cell surface. They are called T-cells because they mature in the thymus (although some also mature in the tonsils). There are several subsets of T-cells, each with a distinct function.

As used herein, the term “B-cell” or “B-lymphocytes” refer to a type of lymphocyte in the humoral immunity of the adaptive immune system. B-cells can be distinguished from other lymphocytes, such as T-cells and natural killer cells (NK cells), by the presence of a protein on the B-cells outer surface known as a B-cell receptor (BCR), allowing a B-cell to bind to a specific antigen. The principal functions of B cells are to make antibodies against antigens, to perform the role of antigen-presenting cells (APCs), and to develop into memory B cells after activation by antigen interaction.

As used herein, the term “NK cell” or “natural killer cell” refers to a type of cytotoxic lymphocyte critical to the innate immune system. The role NK cells play is analogous to that of cytotoxic T-cells in the vertebrate adaptive immune response. NK cells provide rapid responses to virally infected cells and respond to tumour formation, acting at around 3 days after infection. Typically immune cells detect major histocompatibility complex (MHC) presented on infected cell surfaces, triggering cytokine release, causing lysis or apoptosis. NK cells are unique, however, as they have the ability to recognize stressed cells in the absence of antibodies and MHC, allowing for a much faster immune reaction. They were named “natural killers” because of the initial notion that they do not require activation in order to kill cells that are missing “self” markers of major histocompatibility complex (MHC) class 1.

As used herein, the term “cytokine” refers to a broad and loose category of small proteins (˜5-20 kDa) important in cell signalling. They are released by cells and affect the behaviour of other cells, and sometimes the releasing cell itself. Cytokines include chemokines, interferons, interleukins, lymphokines, tumour necrosis factor but generally not hormones or growth factors. Cytokines are produced by broad range of cells, including immune cells like macrophages, B-lymphocytes, T-lymphocytes and mast cells, as well as endothelial cells, fibroblasts, and various stromal cells. A given cytokine may be produced by more than one type of cell. Cytokines modulate the balance between humoral and cell-based immune responses, and also regulate the maturation, growth, and responsiveness of particular cell populations. Some cytokines enhance or inhibit the action of other cytokines in complex ways and are different from hormones, which are also important cell signalling molecules, in that hormones circulate in much lower concentrations and hormones tend to be made by specific kinds of cells.

As used herein, the term “cytokine signalling” refers to the signalling process, whereby each cytokine has a matching cell-surface receptor. Subsequent cascades of intracellular signalling then alter cellular functions. This may include the upregulation and/or downregulation of several genes and their transcription factors, resulting in the production of other cytokines, an increase in the number of surface receptors for other molecules, or the suppression of their own effect by feedback inhibition. The effect of a particular cytokine on a given cell depends on the cytokine, its extracellular abundance, the presence and abundance of the complementary receptor on the cell surface, and downstream signals activated by receptor binding; these last two factors can vary by cell type. Cytokines are characterized by considerable “redundancy”, in that many cytokines appear to share similar functions.

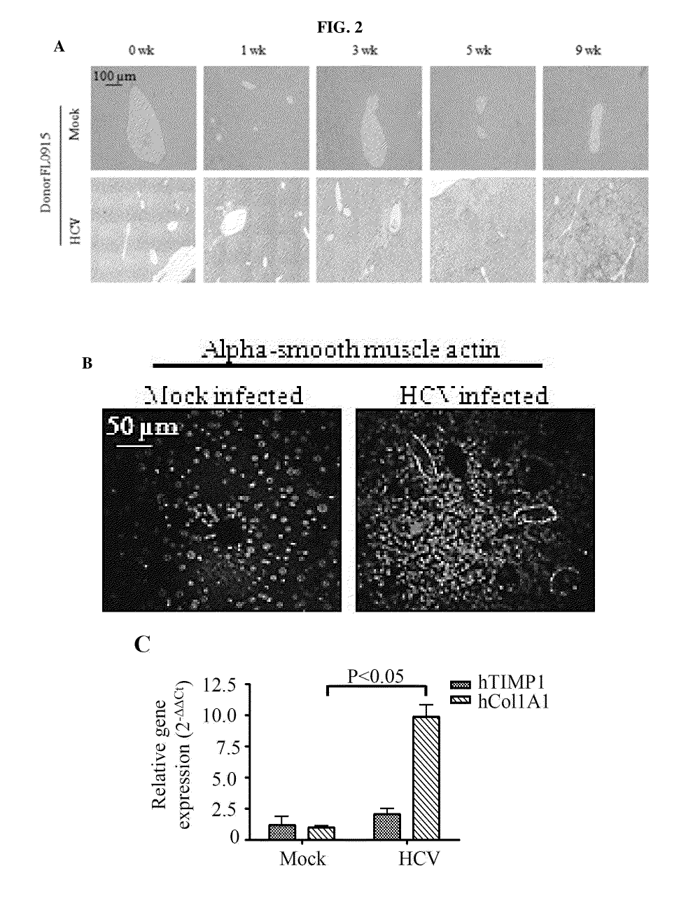

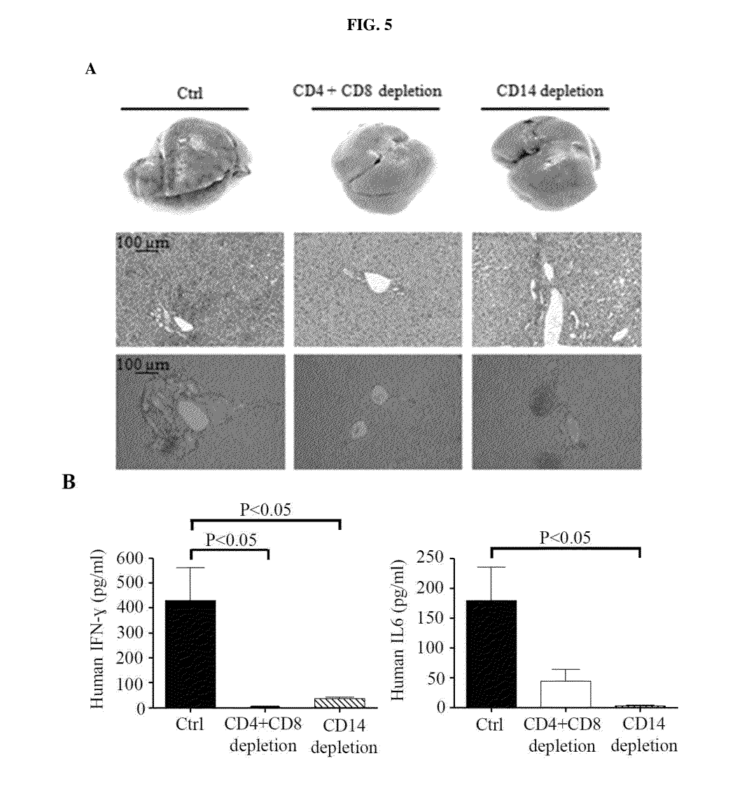

Previous Alb-uPA/SCID or Fah-Rag-γC-null mouse models transplanted with human hepatocytes were able to support HCV infection but did not develop liver fibrosis, mainly due to the absence of a functional immune system. The AFC8-HSC/Hep Balb/C Rag2-γC-null mouse model requires additional drug treatment, possibly making the entire process cumbersome and may interfere with the analysis of markers for liver damage. In addition, the model has not been validated for human hepatoma development resulting from long-term infection. The mouse model present herein largely overcomes all the drawbacks inherent to the existing assays. Firstly, without the need to induce murine hepatocyte apoptosis, the mice as described herein have an increased survival rate and life-span upon human cell transplantation. This omission of intentional apoptosis also allows the production of a large cohort of humanized mice for research and drug testing. Secondly, the platform uses the NSG mouse as a basis which allows efficient human cell engraftment by being able to inject fewer human hematopoietic stem cells and hepatic progenitor cells, e.g. 2×10⁵ CD34+ cells in the mouse model, as described herein, versus 0.5-1×10⁶ CD34+HSCs plus 0.5-1×10⁶ hepatic progenitor cells in AFC8-HSC/Hep model. Thirdly, the mice as described herein consistently develop severe liver damage and seem to have a predisposition to developing hepatocytomegaly, and eventually human hepatoma upon long-term HCV infection (˜6 months). This fact may be linked to the genetic background of mouse strains, which would be worthwhile to study in the future. Another fact is that, although the activation of human immune system has led to the damage of both of human and murine hepatocytes (FIG. 2C), eventually only human albumin positive hepatocytes show hepatocytomegaly and human hepatoma after long-term HCV infection (FIG. 6).

As used herein, the term “long-term” refers to something occurring over or involving a relatively long period of time. This period of time is defined based on the situation at hand and relative to similar situations, e.g. a long-term viral infection with HIV is considered to be years, whereas a long-term infection with an Influenza virus is generally considered to be one month.

As used herein, the term “severe” refers to the intensity (severity) of a specific event or illness, as in mild, moderate or severe. The degree of illness and risk of disease manifested by patients, based either on clinical data and outcome comparisons are usually interpreted in terms of severity of illness to ensure meaningful data interpretations are made.

This de novo development of human hepatocytes in the murine liver represents an approach to repopulate human parenchymal cells in situ in mice. This platform facilitates basic studies of human hepatitis and evaluation of anti-hepatitis therapeutics in the presence of a matching human immune system. In the present disclosure, it is shown that the mice, as described herein not only support HCV infection to induce liver inflammation, HCV-specific human immune responses, liver fibrosis and cirrhosis, but are also able to develop HCV-associated human hepatomas.

As used herein, the term “de novo” refers to something being anew, afresh, from the beginning; without consideration of previous instances, proceedings or determinations.

As used herein, the term “in situ” refers to something that is in its original position or place.

As used herein, the term “in vivo” refers to something being or taking place within a living organism.

In the present disclosure; the widely utilised laboratory HCV isolate, FL-J6/JFH, and serum from a HCV patient were used to infect the mice, as described herein, with both modes of infection yielding similar results. While the level of viral RNA in the blood of the infected mice was too low to be detected, the expression of the HCV core protein was clearly observed in the human hepatocytes in the liver. As controls, viral proteins were not detectable in the HCV-infected. NSG, Balb/c or cord blood-reconstituted (CB) mice, as well as mock-infected mice as described herein. A similar observation had been reported using the AFC8-HSC/Hep model. While no viremia was observed, viral RNA was detected in the liver from 50% of mice at 1 to 4 months post-infection. (FIG. 20) In contrast, significant viremia was detected in the human liver-uPA-SCID or Fah-Rag2γC null mouse models. A possible explanation for this difference is that the level of human hepatocyte engraftment in mice as described herein is only ˜10%, compared to >50% in the uPA or Fah-Rag2γC null mouse models.

In human patients, it is also common to find occult HCV infections, which are defined as the presence of HCV RNA in liver and in peripheral blood mononuclear cells (PBMCs) in the absence of detectable viral RNA in serum by standard assays. Patients with occult HCV viremia were also known to the art to develop liver inflammation and fibrosis. Hence, the absence of viremia in the mouse model, as described herein, does not affect disease development, provided a successful infection is present. In view of the relative low reconstitution of human hepatocytes in mice, as described herein, the results also suggest that an infection in only a small portion of liver cells is sufficient to induce severe disease outcome. In addition to the detection for the presence of HCV core proteins in the human hepatocytes, it was also shown that a specific human T-cell response against the HCV core antigen exists, hence suggesting the presence of latent. HCV infection in the infected mice, as described herein.

The mediators and factors associated with liver disease progression are poorly understood due to a lack of an animal model. The mouse, as described herein, mimics HCV infection occurring in human patients by displaying intrahepatic human immune cell infiltration, HCV-specific human immune responses, as well as liver fibrosis and cirrhosis. Most importantly, this mouse model is able to recapitulate the entire disease progression observed in human HCV patients, i.e. from the early signs of liver damage caused by infection and immunopathogenesis to the eventual development of liver cancer. This makes it possible to study the role of human immune system during hepatitis virus infection and in overall disease progression in the mouse model, as described herein. It was shown that cord blood (CB) reconstituted NSG mice, which are only engrafted using human immune cells isolated from cord blood, but without human hepatocytes, did not support HCV infection and thus were not useful in the observation of liver inflammation or fibrosis. It was also proven that the human immune system, especially T-cells and macrophages, is critical for the development of severe liver diseases, i.e. fibrosis and cirrhosis. This was accomplished by developing a long-term antibody-based in vivo cell depletion method in humanized mice. Both the human T-cells and macrophages contribute to the liver fibrosis, but T-cells seem to be the principal cells responsible for the liver disease development, as the depletion of this cell population results in the complete absence of fibrosis in the mouse. These observations confirm the importance of the presence of both the human immune cells and human hepatocytes to support HCV infection and establish its associated liver disease pathologies in the mouse model.

HCV Chronic Infection in Mice, as Described Herein, Induces Intrahepatic Human Immune Cell Infiltration and Cytokine Responses

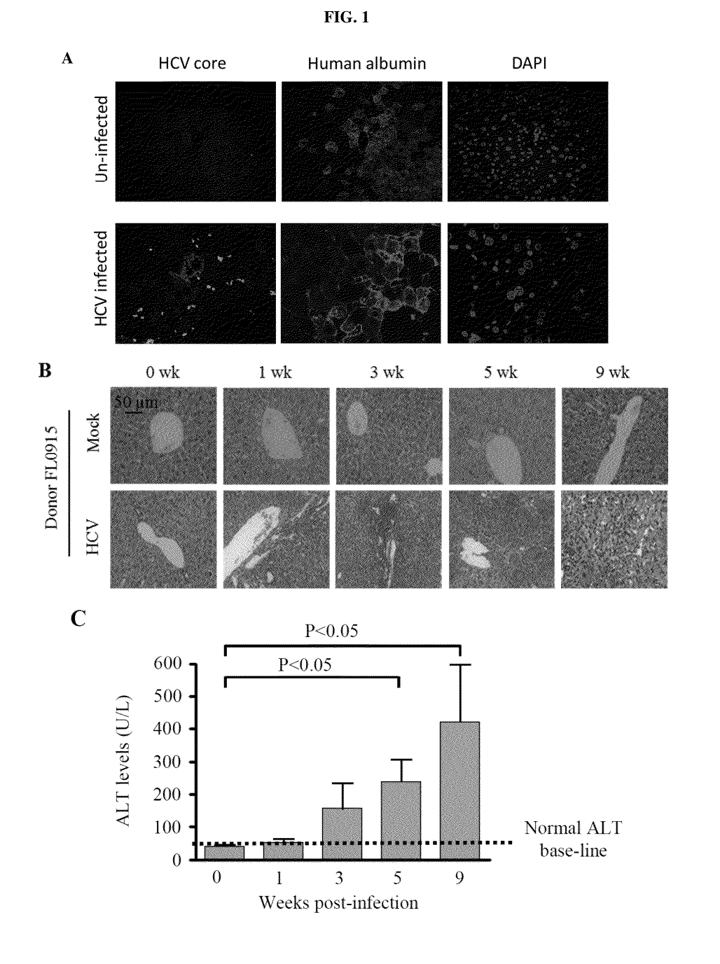

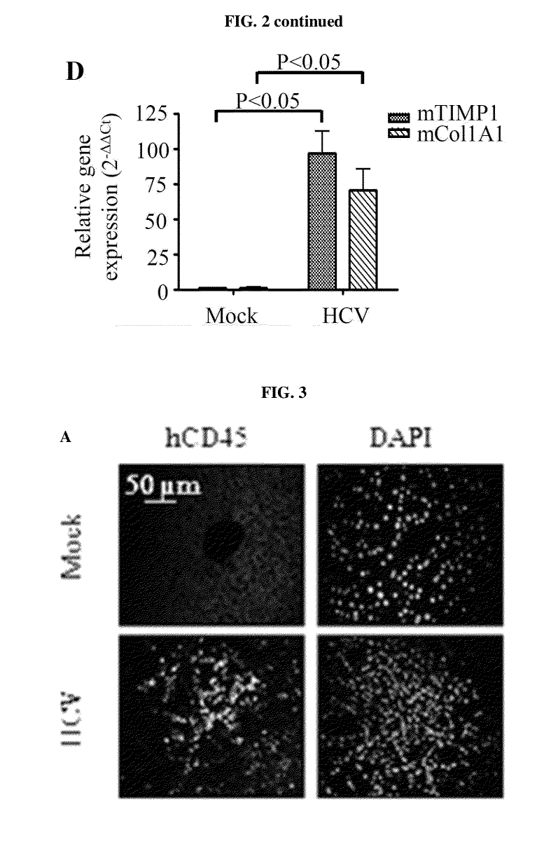

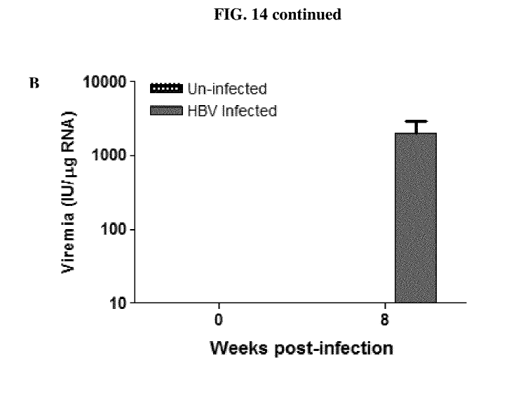

In mice, as described herein, infected with HCV for 9 weeks or longer, liver sections were stained with anti-human CD45 antibodies, showing extensive, infiltration of human CD45+ cells into the liver as early as 5 weeks post-infection (FIG. 3 and FIG. 15) Intrahepatic leukocyte analysis were carried out by isolation of liver mononuclear cells (MNCs), followed by antibodies staining and FACS analysis, i.e. total intrahepatic leukocytes were isolated from the livers of the lab strain HCV-infected mice, as described herein, and stained with 7AAD, anti-CD45-PerCP-Cy7, anti-CD3-APC and anti-CD14-FITC and analysed using flow cytometry. A gradual increase in CD45+ cells in the liver of 3, 5 and 7 weeks HCV-infected mice, as described herein, was observed, while a nearly 3-fold increase in the level of CD45+ cells was observed in the livers of 9 week HCV-infected mice as described herein (FIG. 3B). Among these infiltrated human cells, hepatic human T-cell (CD45+CD3+) populations in HCV-infected mice gradually increases from 3 to 9 weeks post-infection (FIG. 3C). Liver macrophage (CD45+CD14+) cell count increased gradually, reaching a peak at 7 and 9 weeks post-infection (FIG. 3D). The course of human cell infiltration was correlated with the severity of liver damage (FIGS. 1B and 1C), and it was seen that fibrosis and cirrhosis developed at later time points (FIG. 2A), suggesting that human immune system is involved in the disease development. In the art, similar infiltrations have been observed in HCV patients. The total cell count of the leukocyte cells showed that there is only a mild increase of total intrahepatic CD45+ cells from week 1 to week 7 post-infection while more than two-fold increase was observed at week 9 and 11 post-infection, compared with week 0 infected mice (FIG. 3B). A gradual increase in both the CD45+CD3+ human T-cells and the CD45+CD14+ human macrophages in the chimeric mouse liver was also observed, with the increase in T-cells population being observed as early as 1 week post-infection (FIGS. 3C and 3D).

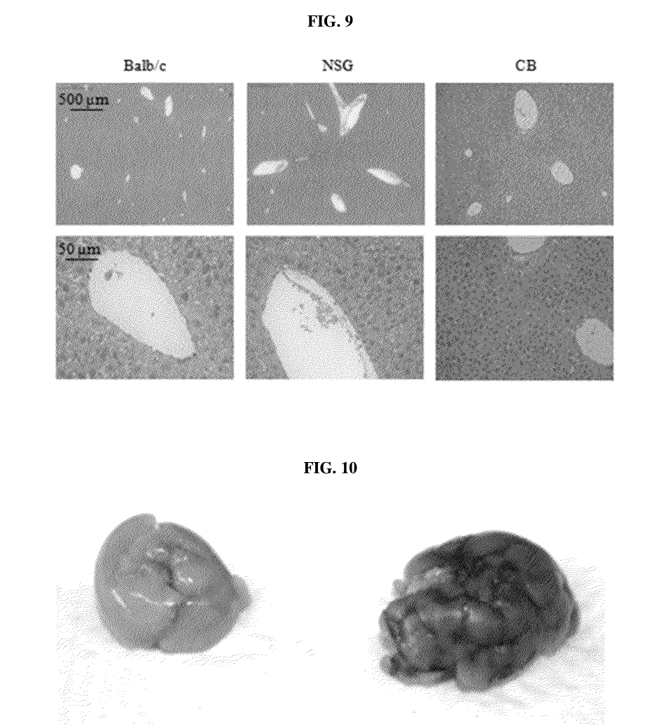

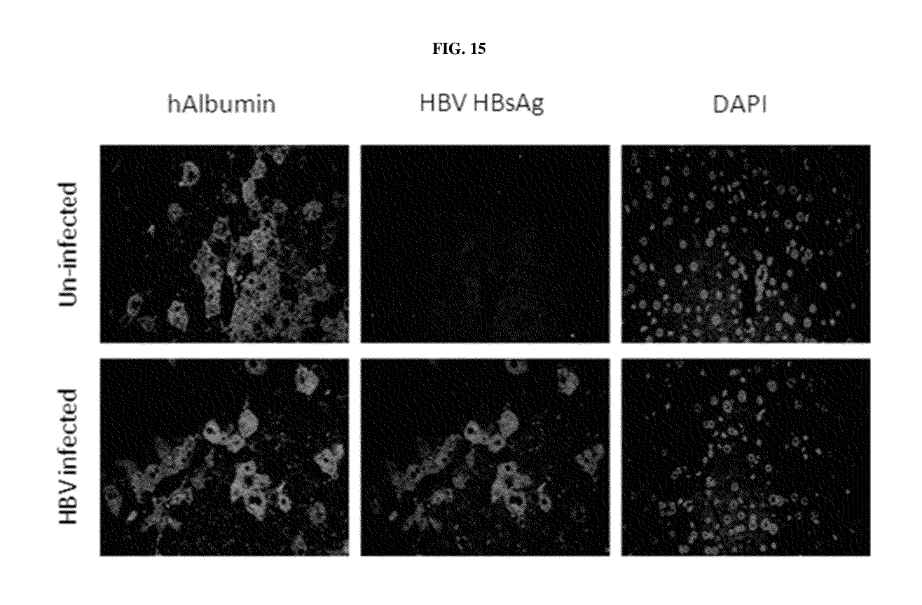

To assay human cytokine response to HCV infection in mice, as described herein, serum human IFN-γ and IL-6 levels were monitored over the course of infection using ELISA kits. Sera were harvested from said mice that had been infected for 0, 1, 3, 5, 7 and 9 weeks and human cytokine levels were determined by ELISA. Both human IFN-γ (FIG. 3E) and IL-6 (FIG. 3F) levels increased gradually over the course of infection but were more significantly elevated at 9 weeks post-infection, with the lab strain HCV, with interferon gamma being detected earlier and at a higher level than IL-6. Human IFN-γ and IL-6 levels were barely detectable over the course of HCV infection in cord-blood reconstituted mice (FIG. 11). Both the interferon gamma and IL-6 cytokine levels peaked at 9 to 11 weeks post-infection at a mean level of about 49 6 ng/ml and 247 ng/ml, respectively (FIGS. 3E and 3F). Only low levels of human interferon gamma and no human IL-6 were detected in the sera of infected cord-blood reconstituted mice. For the clinical strain, both human interferon gamma and IL-6 cytokine responses can be detected with no obvious trend over the course of infection (FIG. 16). The results point to the involvement of human T-cells and macrophages in the HCV response since human interferon gamma was mainly produced by human T-cells while human IL-6 was mainly secreted by human macrophages. Similar immune cell infiltration in the livers has been reported in the art in HCV-infected human patients. Infected cord blood reconstituted mice, without human hepatocytes, did not have significantly elevated human interferon gamma and IL-6 response. Thus, it was shown that the mice, as described herein, were able to support HCV infection in the chimeric liver, leading to leukocyte infiltration of the liver, inflammatory cytokine elevation and liver injury.

These results show that massive human cellular and cytokine responses were induced responding to HCV infection in mice as described herein.

As used herein, the term “CD14” refers to the cluster of differentiation (“CD”) 14. The term “+” denotes the presence of the cluster, whereas “−” denotes the absence thereof. CD14 is expressed mainly by macrophages and (at 10-times lesser extent) by neutrophils. It is also expressed by dendritic cells. The soluble form of the receptor (sCD14) is secreted by the liver and monocytes and is sufficient in low concentrations to confer LPS-responsiveness to cells not expressing CD14. Hence, targeting this CD is used to differentiate macrophages from other cell types.

HCV Infection in Mice, as Described Herein, Elicits Virus-Specific Human T-Cell Response

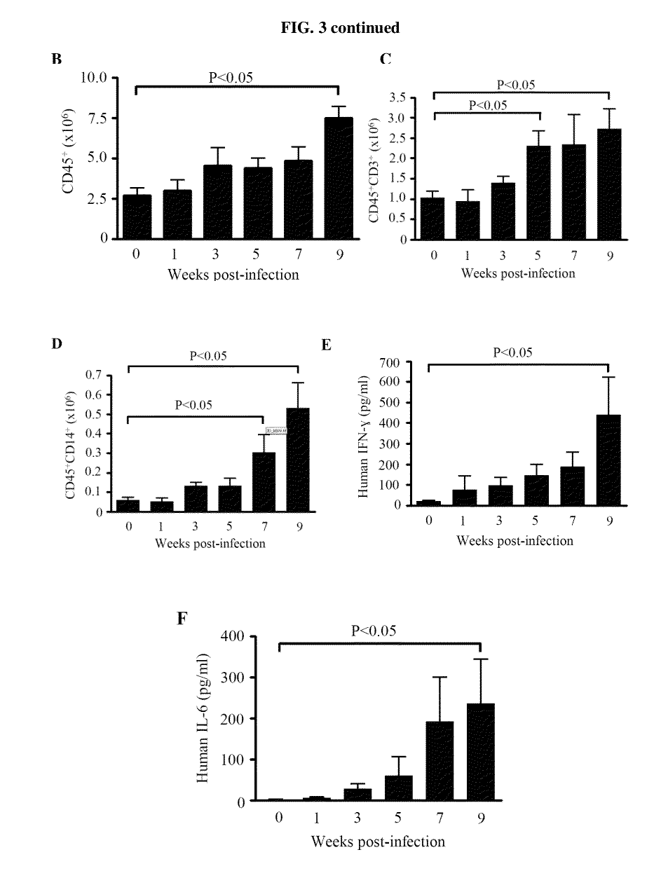

To investigate the presence of human HCV-specific memory T-cell responses, splenocytes from HCV-infected mice as described herein were harvested at 9 weeks post-infection and used in human IFN-γ ELISpot assays. Mock-infected mice, as described herein. were included as negative controls. Freshly isolated splenocytes were stimulated directly ex vivo for 48 hours using a mixture of 16 synthetic peptides that overlap with the HCV core protein. Positive controls consist of splenocytes stimulated with PMA and ionomycin (FIG. 4A, last column). No human T-cell response was detected in mock-infected mice with or without peptide stimulation (FIG. 4A, bottom row). The number of IFN-γ producing spots forming units (SFU) were detected at higher frequencies in wells containing peptide-stimulated splenocytes from HCV-infected mice, with a mean of 485, compared to mock-stimulated splenocytes from HCV-infected mice, which had a mean of 50 (FIG. 4B). This result reveals that HCV-specific memory T-cells are present in HCV-infected mice as described herein.

In one example, the present disclosure describes a mouse as defined herein, wherein the Prkdc-gene of the mouse contains a mutation that results in a loss-of-function of the Prkdc gene, thus resulting in the presentation of a severe combined immunodeficiency (scid). This mutation is also known as Prkdc^{scid}.

As used herein, the term “Prkdc” refers to a gene known to express DNA-dependent protein kinase, catalytic subunits, also known as DNA-PKcs. These DNA-PKcs are enzymes that belong to the phosphatidylinositol 3-kinase-related kinase protein family. DNA-PKcs, along with their second component, the autoimmune antigen Ku, is required for the non-homologous end joining (NHEJ) pathway of DNA repair, which rejoins double-strand breaks. It is also required for V(D)J recombination, a process that utilizes NHEJ to promote immune system diversity. DNA-PKcs knockout mice are known to have severe combined immunodeficiency due to their V(D)J recombination defect.

As used herein, the term “scid” stands for severe combined immunodeficiency and is defined as a severe immunodeficiency genetic disorder that is characterized by the complete inability of the adaptive immune system to mount, coordinate, and sustain an appropriate immune response, usually due to absent or atypical T and B lymphocytes.

As used herein, the term “Prkdc^{scid}” refers to a loss-of-function mutation in the Prkdc gene, which commonly results in a severe combined immunodeficiency (scid), As a result, mice which are homozygous for this mutation have severely reduced numbers of mature T and B cells. The phenotypic penetrance of Prkdc^{scid} varies among murine inbred strain backgrounds, but the mutation is most effective at eliminating adaptive immunity in mice with the so-called non-obese diabetic (NOD) genetic background.

HCV Infection of Mice as Described Herein Leads to Leukocyte Infiltration and Lesions in the Liver

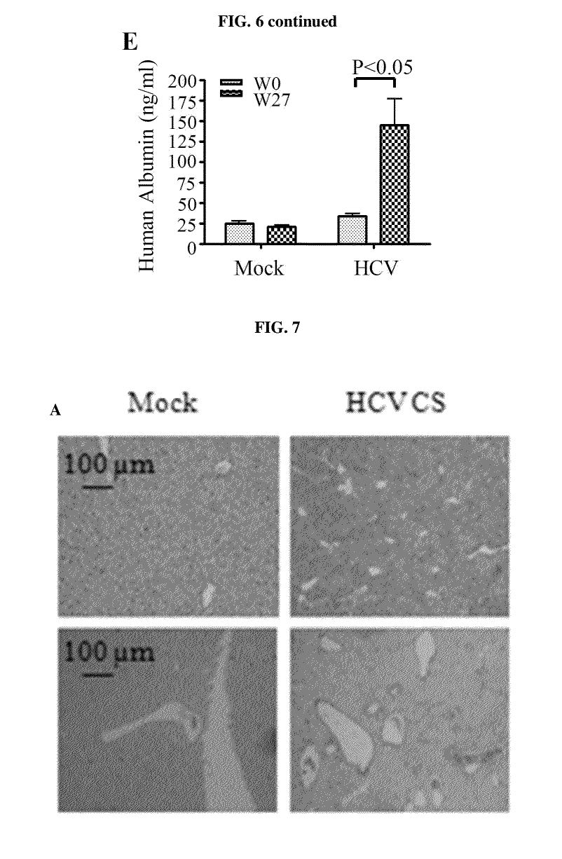

CD34+ cells isolated from fresh human fetal livers, which had been collected within half-hour after abortion and were used as sources of progenitors for both hematopoietic stem cells (HSCs) and hepatic stem cells (HPCs). Mice, as described herein, were generated using adoptive transfer of CD34+ cells into sub-lethally irradiated, new-born NSG pups. In total, samples from 12 different donors were used to generate mice as described herein. Eight weeks after HSC transfer, peripheral blood mononuclear cells (PBMCs) of recipient mice were stained for human and murine CD45 and analysed using a fluorescence-activated cell-sorting (FACS) to determine the reconstitution level of human leukocytes [% human CD45+ cells/(% human CD45+ cells+% mouse CD45+ cells)] (FIG. 8A). The mice showed good reconstitution of human immune cells, with a mean percentage of about 40% (FIG. 8B). Human albumin level in the serum of the mice were analysed using a human albumin ELISA kit. The mean level of human albumin in the mice is determined to be about 24 ng/ml (FIG. 8C). Liver sections from NSG and mice as described herein were stained for human albumin. There was no positive staining seen for human albumin in the liver of the NSG mice, but a significant proportion, 5-15% of the hepatocytes in the liver of mice as described herein, were positively stained (FIGS. 8C and 8D). Sera from eight-week-old mice, as described herein, were tested for human albumin levels using ELISA. The results showed that all the reconstituted mice had human albumin in their serum, with a mean value of 26.4 ng/ml (FIG. 8D).

Ten-week old mice, as described in the present description, were inoculated with 1×10⁶ focus forming units (FFU)/ml of J6/JFH-1 HCV (genotype 2a), or with clinical strains (genotype 3a, 4×10⁵ IU) of HCV viruses or mock infected via intravenous tail-vein injection. Based on genetic differences, the HCV viruses are divided into different genotypes. Different genotypes of HCV result in different clinical outcomes and responses to therapy. The laboratory strain J6/JFH-1-P-47 is a culture-adapted variant of J6/JFH-1 obtained by serial passaging of J6/JFH1-infected Huh7.5 cells. Detailed description of the parental J6/JFH1 virus and the Huh7.5 cells are provided in Lindenbach et al. (Lindenbach, B. D., Evans, M. J., Syder, A. J., et al.; Science (2005) 309, 623-626). In one example, the present description provides a mouse as described herein, wherein the process further comprises injecting hepatitis B virus (HBV) or hepatitis C virus (HCV) into the mouse. Each mouse infection group was injected with only one viral agent.

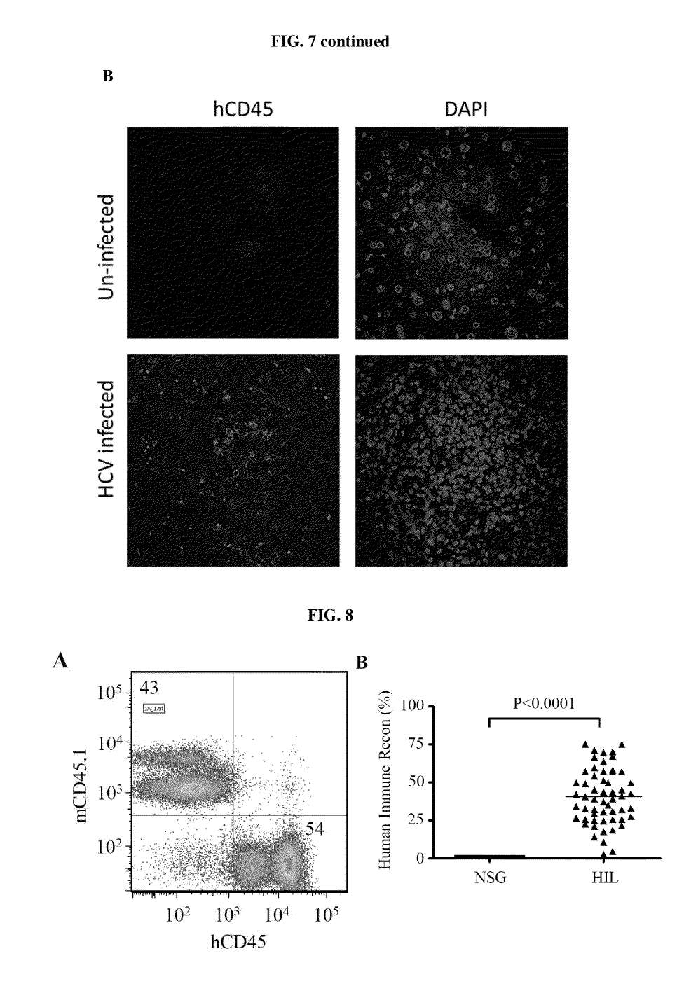

Livers of the infected mice, as described herein, were collected for histology sections at weeks 0, 1, 3, 5, 7, 9 and 11. Specific staining for the HCV core protein showed a co-localization with human albumin staining in the lab strain HCV-infected mice, as described herein, liver sections, as early as 5 weeks post-infection (FIG. 13) and at 11 weeks post-infection in liver sections of the clinical strain HCV-infected mice, as described herein (FIG. 15). While antibodies against HCV core antigen were shown to co-localise with antibodies against human albumin in the liver sections of the infected mice, as described herein, this was not seen in the mock-infected mice (FIG. 1A). This result suggests that HCV is able to infect and express viral antigens in human hepatocytes of infected mice, as described herein. For the clinical strain, the leukocyte infiltration in the liver and liver injury are observed at 11 weeks post-infection (FIG. 15). In liver sections of mice as described herein that had been infected for 0, 1, 3, 5 and 9 weeks, an increasing leukocyte infiltration and progressive lesions formation over the course of the infection was observed via Hematoxylin and Eosin (H&E) staining (FIG. 1B). This was not observed in Balb/c mice with a murine immune system and hepatocytes, or in cord-blood (CB) reconstituted NSG control mice without human cell reconstitution (FIG. 9). Moreover, it had been shown that NSG mice injected with human cord blood CD34+ cells only developed a human immune system, but no human hepatocytes. Furthermore, infection of these cord blood CD34+ cell reconstituted mice with HCV showed no development of infiltration and lesions in the liver (FIG. 9). These results indicate that both the human immune system and hepatocytes are required to recapitulate the liver pathogenesis observed in HCV patients. Consistent with the progressive liver damage observed in H&E staining, serum Alanine transaminase (ALT) levels were shown to be elevated over the course of HCV infection in the mice, as described herein (FIG. 1C). Elevated ALT levels are widely used in the art in clinical settings as a marker for liver damage during HCV infection Summarizing the above, mice as described herein are able to support HCV infection in human hepatocytes of chimeric livers, which leads to immune cell infiltration and progressive liver damage displaying elevated serum ALT levels.

Hence, the present description describes herein the mouse, as defined herein, wherein the CD34+ stem cells are obtained from human fetal tissue. In another example, the CD34+ stem cells are obtained from human fetal liver. In a further example, CD34+ stem cells used for injection are obtained from freshly harvested human fetal liver. In another example, the CD34+ stem cells are not obtained from frozen human fetal liver. In a further example, freshly harvested human fetal liver is between about 15 minutes to 2 hours old, or between about 30 minutes to 1.5 hours old, or between about 30 minutes to 1 hour old, or about 1 hour or 30 minutes or 45 minutes or 20 minutes old. In another example, the CD34+ stem cells comprise progenitors of the human hepatocytes. In further example, the CD34+ stem cells essentially consist of or consist of CD133(hi) hematopoietic stem cells or consist of and CD133(lo) hepatic stem cells.

As used herein, the terms “CD133(hi)” and “CD133(lo)” refer to the staining of cells, in this case with CD133, and the resulting FACS signal measured, with (hi) implying a strong binding of the staining agent to the cell and (lo) denoting a weak binding of the staining agent to the cell. In this description, CD133(hi) refers to CD34+ stem cells that were stained strongly for the marker CD133 in FACS analysis. In the same line, CD133(lo) are CD34+ stem cells that were stained weakly for the marker CD133 in FACS analysis.

In another example, the CD34+ stem cells have been injected by one-step injection. In a further example, the entire population of CD34+ stem cells isolated from human fetal liver is delivered into the mouse.

The present disclosure also provides a mouse for human hepatitis studies obtained by a process of injecting CD34+ stem cells into an immunocompromised mouse. In one example, the CD34+ stem cells are as defined herein. In a further example, the CD34+ stem cells are injected into a 1 to 3 day old mouse.

As used herein, the term “essentially” or “essential” refers to a descriptive term of something being in the basic form, i.e. showing its essence, and is used to emphasize the basic, fundamental, or intrinsic nature of a person or thing.

Human T-Cells and Macrophages Play Critical Roles in Inflammation, Fibrosis Development and Human Cytokine Response in HCV-Infected Mice as Described Herein

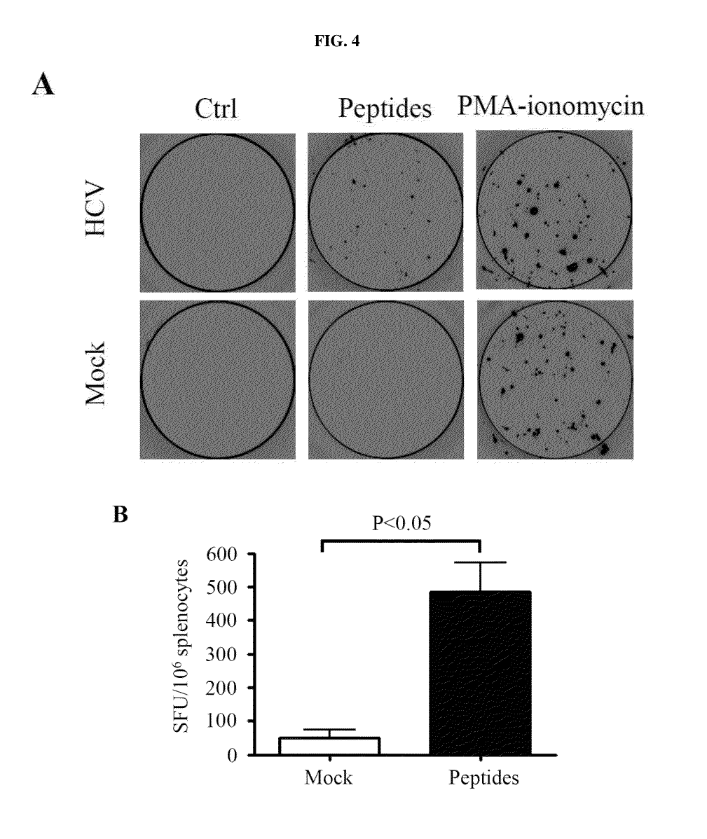

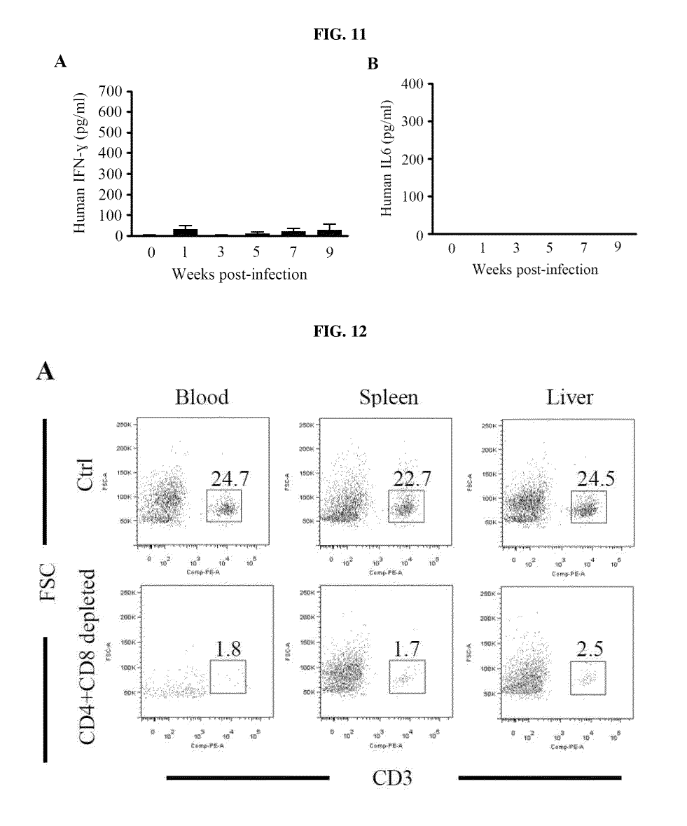

To investigate the roles of specific human immune cell populations in the cytokine response, leukocyte infiltration and fibrosis during HCV infection, an antibody-induced depletion of human T-cells and macrophages in mice as described herein was preformed prior to HCV infection tail vein injection of 50 μg of CD4+CD8 antibodies or CD14 antibodies. As anti-CD3 antibodies are known in the art to activate T-cells and lead to non-specific cytokine release, serial injection of anti-human CD4 and CD8 antibodies into mice, as described herein, were performed, i.e. regular injection of 20 μg of the specific antibodies every 3 to 4 days throughout the course of infection, in an attempt to achieve long-term depletion of the CD3+ T-cells. For the depletion of CD14+ macrophages, anti-human CD14 antibody was used. The effect of systematic targeted cell depletion is shown (FIGS. 3 and 12, respectively). At 5 weeks post-infection, a significant decrease in the human interferon response between the undepleted mice and the CD45+CD3+ depleted mice (FIG. 5B) was observed. A slight decrease in the human interferon response in the CD45+CD14+ depleted mice was also observed (FIG. 5B). The result confirmed that the CD45+CD3+ cells were responsible for human interferon response during HCV infection. Eight weeks after HCV infection, extensive liver scarring with leukocyte infiltration and fibrosis could be observed in PBS-treated mice (ctrl) (FIG. 5A, first column). For mice, as described herein and that had been depleted of CD4 and CD8 T-cells, no visible liver scarring, leukocyte infiltration or fibrosis was observed after HCV infection (FIG. 5A, middle column). When mice as described herein were depleted of CD14 macrophages, a significant reduction of liver scarring with limited leukocyte infiltration and slight collagen deposition was observed (FIG. 5A, last column).

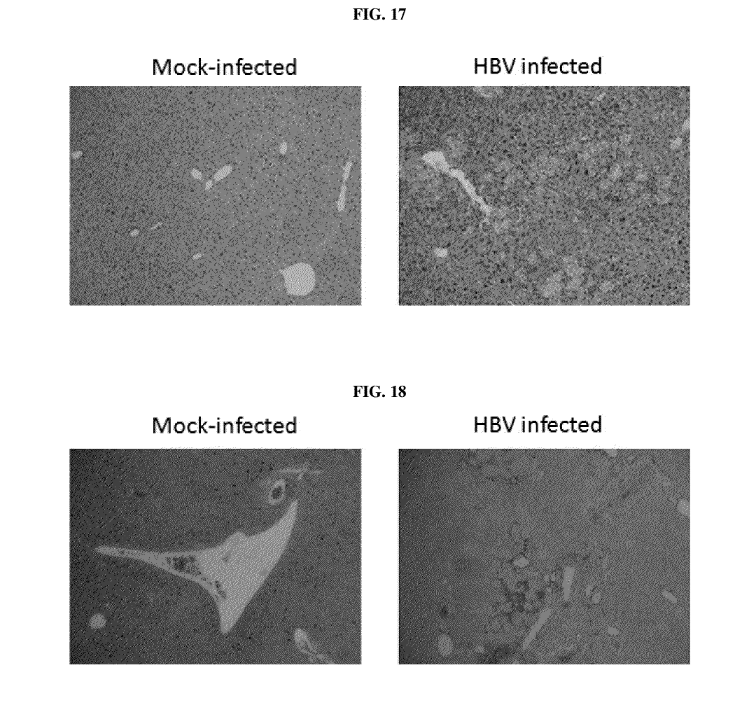

The mice as described herein were also inoculated with culture supernatant of HBV (un-titrated). Livers of the infected mice were collected for histology sections at weeks 5, 7 and 9. Specific staining for the HBV Pre-S2 antigen were detected in the infected mice liver sections at 7 and 9 weeks post-infection (FIG. 18). Elevated human interferon gamma levels were detected in the sera of week 5, 7 and 9-infected mice, while human IL-6 response was only observed at week 7 and 9 post-infection (FIG. 20).

Serum levels of human IFN-γ were dramatically reduced in the depleted groups, from a mean level of 431.8 pg/ml in control mice to 2 pg/ml in T-cell-depleted mice and 38.8 pg/ml in macrophage-depleted mice (FIG. 5B, left). Serum levels of human IL-6 were reduced from a mean level of 179.8 pg/ml in control mice to 44 pg/ml in T-cell-depleted mice, and 3 pg/ml in macrophage-depleted mice (FIG. 5B, right). These results indicate that both T-cells and macrophages play important roles in the HCV induced liver pathogenesis. The elimination of the CD14+ cells resulting in the retention of some aspects of the liver pathology of HCV infection, whereas the depletion of CD4+ and CD8+ T-cells essentially eliminated the diseased pathology, suggesting T-cells are the principal immune cells contributing to the liver scarring and fibrosis in HCV infection, with a supportive role from macrophages. The depletion of either human T-cells or macrophages resulted in a drastic reduction of human IFN-γ or IL-6 levels, suggesting that there are interplays between the T-cells and macrophages in the immune responses to HCV infection.

Significant serum levels of human cytokine responses were reported in HCV infection model. It had further been shown that human IFN-γ is mainly secreted by activated human T-cells, while human IL-6 is secreted by human macrophages, confirming that both human T-cells and macrophages are critical in immune responses to HCV infection. It was also noted that the depletion of either human T-cells or macrophages resulted in a decrease in the levels of both human IFN-γ and IL-6. These results imply that there is crosstalk present between the human innate and adaptive immune systems during HCV infection. At the present time, the functions of other human immune cells, e.g. human NK cells and B-cells in humanized mouse models are sub-optimal due to some species barriers. However, with the presently developed methods to improve human immune system in mice, a deeper understanding of the underlying mechanisms of human immune responses, e.g. anti-viral NK cell responses, HCV specific antibody response etc. during HCV infection, using the mouse model as described herein, is expected.

Hence, the present description provides for a mouse as disclosed herein, wherein the mouse is capable of developing one or two or three or four or five or six conditions selected from the group consisting of human leukocyte inflammation, human immune responses, liver damage, such as cirrhosis and fibrosis. In one example, the mouse is capable of developing human hepatocellular adenoma and human hepatocellular carcinoma after long term HCV infection. In a further example, the mouse is not genetically modified to allow HCV infection.

HCV Infection of Mice as Described Herein Leads to Activation of Hepatic Stellate Cells and Up-Regulation of Fibrotic Genes with Consequential Liver Fibrosis and Cirrhosis

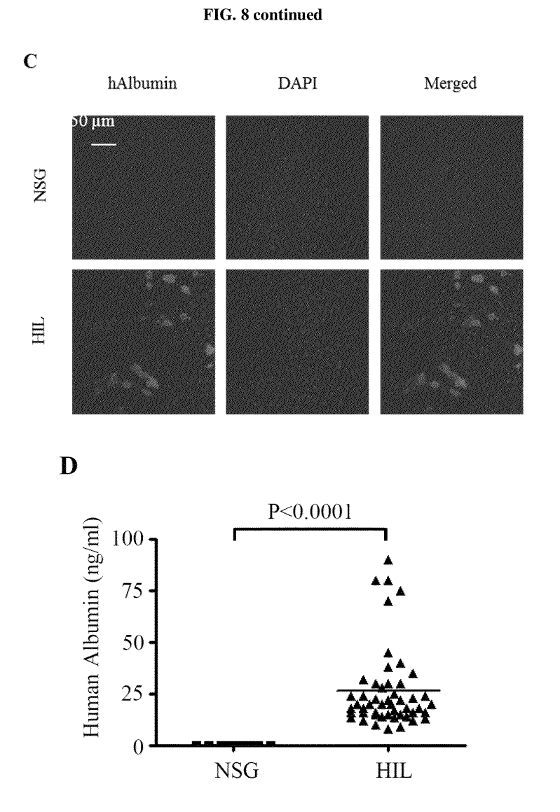

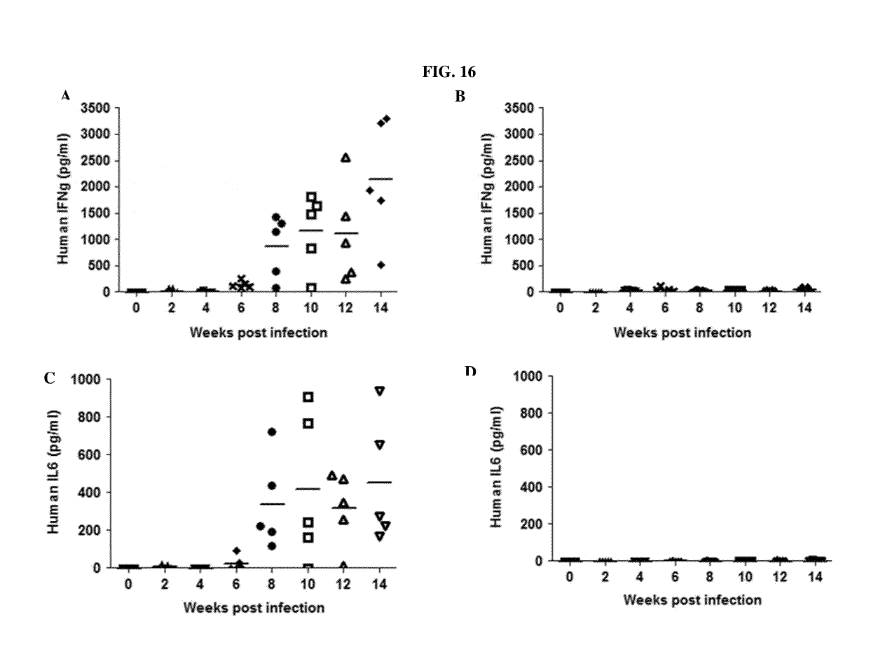

In human patients, chronic HCV infection often leads to severe liver pathogenesis such as fibrosis and cirrhosis. To assess the progression of liver pathology of the infected mice as described herein, the livers of week 1, 3, 5 and 9 post-infected mice were harvested for histological examination. Nodule fibrosis was observed in the livers of mice at 3 weeks post-infection (FIG. 2A). Fibrosis with numerous septa was present in the livers of 5 weeks post-infected mice (FIG. 2A). Finally, cirrhosis with regenerative nodules was observed in the livers of mice that had been infected for 9 weeks or more (FIG. 2A). A representative picture of a cirrhotic liver with severe scarring and nodule formation is shown in FIG. 10, right. Mock-infected mice, as described herein, and HCV-infected NSG mice that were reconstituted with only the human immune system did not develop the pathology. Balb/c mice with a fully functional mouse immune system and mouse hepatocytes also did not develop fibrosis. For clinical strain infected mice as described herein, fibrosis is detected in the liver at 11 weeks post-infection (FIG. 17). The activation of hepatic stellate cells during HCV infection is commonly detected in HCV patients, which contributes to liver fibrosis. It was possible to detect specific staining for the activated hepatic stellate cells by immunostaining of the liver sections of lab strain HCV infected mice as described herein, using anti-alpha-smooth muscle actin (αSMA) monoclonal antibody (FIG. 2B, right). The uninfected control mice showed negative staining for αSMA (FIG. 2B, left). Tissue inhibitor of metalloproteinases-1 (TIMP1) has been shown to contribute to fibrosis by attenuating the degradation of extracellular matrix and fibrosis resolution.

HCV infection in human patients has been known to lead to the activation of hepatic stellate cells, which are known in the art to contribute to disease progression to fibrosis. Liver sections from 9 weeks post-infected mice as described herein were stained with anti-alpha smooth muscle actin monoclonal antibodies to detect activated hepatic stellate cells. The result showed the presence of activated hepatic stellate cells in the liver of the HCV-infected mice (FIG. 2B, right) but not in the liver of the mock-infected mice (FIG. 2B, left). Gene expressions of collagen 1A1 (Col1A1) and tissue inhibitor of matrix metalloproteinases 1 (TIMP1) are known to be elevated in activated hepatic stellate cells for attenuation of liver fibrosis. Using human-specific and mouse-specific gene primers, the mRNA levels of human and mouse Col1A1 and TIMP1 were analysed, using human and mouse GAPDH, respectively, for normalization purposes. The induction, and thus upregulation, of both the human and mouse Col1A1 and TIMP1 in the livers of the HCV-infected mice, as described herein, was observed (FIGS. 2C and 2D, respectively). These results suggest that the liver inflammation and damage caused by HCV infection can activate hepatic stellate cells to induce fibrosis and cirrhosis in the chimeric liver of mice as described herein, indicated by the expression of both human and murine fibrogenic genes.

Chronic HCV Infection of Mice, as Described Herein, Leads to the Development of Human Hepatoma.

To investigate whether chronic HCV infection in mice as described herein can eventually lead to the development of human hepatoma, livers were harvested from mice as described herein at 27 weeks post-infection for histological analysis. Visible tumorigenic growths were observed in all the livers of the long-term HCV-infected mice (FIG. 6A). Pathological analysis confirmed that the livers have multifocal tumours, most of which are hepatocellular adenomas, composed of nodular growth of bland-appearing hepatocytes with loss of normal architectural patterns, including the absence of portal tracts and central veins (FIG. 6B). The hepatoma produced a slight compression of the adjacent normal hepatic parenchyma and the individual hepatocytes were organized into cords or solid sheaths and often had vacuolated (glycogen or fat) cytoplasm (FIG. 6C). Multifocal areas of necrosis were occasionally seen within the tumour and frequent occurrences of hepatocytomegaly with increased hepatocyte mitosis were also observed (FIG. 6C). To confirm the tumour origin is of human hepatocytes, the liver sections were stained with anti-human albumin antibodies. Positive staining for human albumin was observed in all the tumour growths (FIG. 6D). An eight-fold increase of serum human albumin levels was observed in the same mice 27 weeks after infection compared to week 0 before infection. This increase was not observed in the mock-infected mice. These results show that the HCV-infected mice, as described herein, are able to develop HCV-associated human hepatoma.

Clinical HCV Strain can Re-Produce the Liver Pathogenesis in Mice, as Described Herein

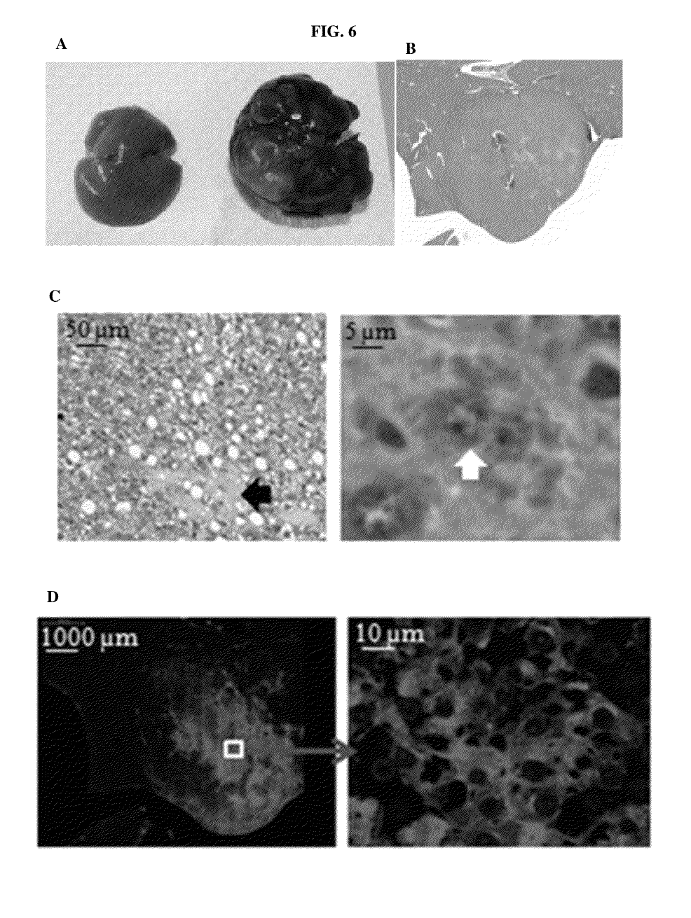

To investigate whether that the mouse model, as described herein, can be used for the study of different genotypes of HCV strains, mice were inoculated with a HCV clinical strain (CS) of genotype 3a. Observed leukocyte infiltration and fibrosis in the HCV CS-infected mice, as described herein, were seen that were similar to that observed using FL-J6/JFH-1 infected mice (FIG. 7A). Because the virus titre of clinical strain used is lower than the lab strain, the disease progression was slower thus fibrosis was observed around 13 to 15 weeks post-infection. The immunostaining of human CD45+ cells confirmed the infiltration of human immune cells, which was not observed in mock-infected mice, as described herein, (FIG. 7B). These data showed that mice, as described herein, can also re-produce liver pathogenesis observed in patients with clinical HCV strains.

Thus, in one example, the mouse is as described herein, wherein said mouse is characterized as defined herein. The present description further provides a method of drug screening, wherein the method comprises administering anti-HBV or anti-HCV therapeutics to a mouse as defined herein. In another example, there is described a method of characterizing changes in viral quasispecies during HBV or HCV infection by using a mouse as defined herein. Furthermore, in another example, there is described a use of a mouse, as defined herein, for testing the efficiency of putative anti-HBV or anti-HCV drugs or for characterizing changes in viral quasispecies during HBV or HCV infection. As used herein, the term “quasispecies”, describes a group of virus that have similar genomes.

In summary, the results above demonstrate that human stem cell engraftment supports the reconstitution of both human immune system and hepatocytes in the mice, as described herein, which provides an ideal platform for in vivo study of hepatotropic pathogens. Hepatitis B virus (HBV) and hepatitis C virus (HCV) infections are supported in this mouse model, and strains of different HCV genotypes can lead to severe liver diseases. Furthermore, robust human immune responses including cytokine responses and antibody responses have been detected in HBV-infected mice as described herein. Immune cells infiltration of the livers and severe fibrosis, which is typical of chronic infection, was consistently detected in infected mice, as described herein. Hence in general, this model as described herein will provide a system for detailed characterization of viral-host interactions, HCV-specific human immune responses and HCV-associated fibrosis, cirrhosis and liver cancer developments using strains of different genotypes. It can also serve as a platform for therapeutic drug screening and vaccine development, as well as the identification of new markers for HCV-associated liver cancer. Besides HCV, it can also be used as a good platform to study other hepatotropic pathogens, such as malaria and hepatitis B virus, as well as HIV/HCV and HBV/HCV co-infections, and function as a model for HBV-induced human carcinoma, as promising results have been shown for the HCV mouse model. The utilized NOD scid gamma (NSG) background allows for engraftment of human immune cells. A simple and efficient method to reconstitute both human immune cells and hepatocytes in the same mouse is provided, making additional transgene, surgery or drug treatment unnecessary. The presented model has been shown to display human immune responses, showing specifically human hepatocyte reconstitution and maturation, which in turn lead to severe fibrosis and cirrhosis.

The invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### Experimental Section

### Human Fetal Liver Progenitor Stem Cells

Human CD34+ cells were freshly isolated from aborted foetuses at 15 to 23 weeks of gestation, in accordance with the institutional ethical guidelines of the KK Women's and Children's Hospital, Singapore. Briefly, fetal liver tissue was cut into small pieces and digested with collagenase IV (1 mg/ml in DMEM) for 15 min at 37° C. on a rotator as described in Chen et al., 2013. CD34+ cells were purified by magnetic-activated cell sorting using the EasySep CD34 positive selection kit (Stemcell Technologies) under sterile conditions, following manufacturer's protocol. The purity of the CD34+ cells was 90 to 99%.

Mice and Transplantation of Human CD34+ Cells

NSG mice were purchased from Jackson laboratory and bred in a specific pathogen-free (SPF) facility at the biological resource centre (BRC) in Agency for Science, Technology and Research (A*STAR), Singapore. Balb/c mice were purchased from the BRC. One to three days old NSG pups were sub-lethally irradiated at 1 Gy and transplanted with 2×10⁵ CD34+ human fetal liver cells by intra-hepatic injections. The mice were bled at 8 weeks post-transplantation to determine the human immune reconstitution level and serum human albumin level. All experimental procedures were in accordance to protocols approved by the International Animal Care and Use Committee (IACUC).

HCV Infection

Ten-week old mice, as described herein, were infected with HCV by intravenous injection of the viruses. 1×10⁶ FFU of a laboratory strain FL-J6/JFH (genotype 2a) or 4×10⁵ virus copies of clinical strain (genotype 3a) were used. For the laboratory strain, a cell culture adapted P-47 was previously generated (Bungyoku et al., 2009) and used throughout the experiments.

Immunohistochemical Staining

At the different time points, HCV-infected/mock-infected mice were sacrificed and the livers were fixed with 10% formalin and embedded in paraffin for processing into liver tissue sections. Rehydrated liver tissue sections were either stained with Hematoxylin & Eosin (H&E) (Thermo Scientific) or Fast-green (Sigma) & Sirius Red (Sigma) or antigen-retrieved using 20 μg/ml proteinase K and stained with anti-human CD45 (ab781, abeam), anti-αSMA (ab5694, abcam), anti-HCV core (MA1-080, Pierce) or anti-human albumin (ab2406, abeam) antibodies. All fluorescence images were acquired using Olympus upright confocal microscope with Fluoview acquisition software and using oil medium with 40× objective lens. All H&E or Fast-green & Sirius Red stained images were captured using MIRAX MIDI fluorescence microscope (Zesis) using the MIRAX acquisition software.

Flow Cytometry

Mononuclear cells (MNCs) were isolated from blood and stained with anti-mouse CD45.1-PE-Cy7 or anti-human CD45-FITC for human reconstitution analysis. MNCs were isolated from livers and stained with 7-AAD, anti-human CD45-PE-Cy7, anti-human CD3-APC or anti-human CD14-PE for intrahepatic leukocyte analysis or stained with 7-AAD, anti-mouse CD45.1-PE-Cy7, anti-human CD45-FITC, anti-human CD3-PE and anti-human MMR-APC for immune cells depletion analysis. All the antibodies for FACS analysis were purchased from Biolegend (San Diego, Calif.). All data were collected using FACSCanto flow cytometer (BD Biosciences) and analyzed using the FlowJo version 7.5.5 software.

Enzyme-Linked Immunosorbent Assay (ELISA)

Blood samples collected from the mice were centrifuged at 3000 rpm for 10 mins at 4° C., and serum samples were harvested for ELISA analysis. Serum human albumin level was determined using the human albumin ELISA kit (Bethyl Laboratories), following manufacturer's protocol. Human serum cytokines level were determined using human IFN-γ ELISA kit (Biolegend) and human IL-6 ELISA kit (Biolegend), following manufacturer's protocol.

### In Vivo Depletion of Specific Immune Cells Population in Humanized NSG Mouse

T-cells and CD14+ cells were depleted from ten-week old mice, as described herein, by intravenous injection of 50 μg of anti-human CD4 plus anti-human CD8 or anti-human CD14 antibodies, respectively. Control mice were injected with PBS instead. Depletion of the specific immune cell population was maintained by intravenous injection of 20 μg of the respective antibodies every 3 days. Depletion efficiencies of specific immune cell population from the blood, liver and spleen of mice, as described herein, using this method were verified by flow cytometry.

Human IFN-γ Enzyme-Linked Immunospot (ELIspot) Assay

Mononuclear cells (MNCs) were isolated from fresh livers of mice, as described herein, that were infected with HCV for 9 weeks by Percoll density gradient centrifugation and re-suspended in AIM-V medium (Invitrogen) with 2% human AB serum, supplemented with interleukin-2 at 10 IU/ml and interleukin-15 at 10 ng/ml. MNCs were seeded at 10⁶ cells/ml/well in 24-well plate and pre-stimulated with and without 10 μg/ml of a pool of 16 overlapping 20-mer peptides (Genscript), covering the HCV core protein, at 37° C. for 24 h. A complete list of the peptide sequences are provided in Table 1 below.

TABLE 1
List of HCV core peptides for stimulation of
T-cells in ELISpot

| Peptide name | Sequence |
|---|---|
| HCV core P1 | MSTNPKPQRKTKRNTNRRPQ |
| HCV core P2 | TKRNTNRRPQDVKFPGGGQI |
| HCV core P3 | DVKFPGGGQIVGGVYLLPRR |
| HCV core P4 | VGGVYLLPRRGPRLGVRATR |
| HCV core P5 | GPRLGVRATRKTSERSQPRG |
| HCV core P6 | KTSERSQPRGRRQPIPKDRR |
| HCV core P7 | RRQPIPKDRRSTGKSWGKPG |
| HCV core P8 | STGKSWGKPGYPWPLYGNEG |
| HCV core P9 | YPWPLYGNEGLGWAGWLLSP |
| HCV core P10 | LGWAGWLLSPRGSRPSWGPN |
| HCV core P11 | RGSRPSWGPNDPRHRSRNVG |
| HCV core P12 | DPRHRSRNVGKVIDTLTCGF |
| HCV core P13 | KVIDTLTCGFADLMGYIPVV |
| HCV core P14 | ADLMGYIPVVGAPLGGVARA |
| HCV core P15 | GAPLGGVARALAHGVRVLED |
| HCV core P16 | LAHGVRVLEDGVNFATGNLP |

Positive control consists of MNCs stimulated with 50 ng/ml Phorbol 12-Myristate 13-acetate (PMA) (Sigma) and 500 ng/ml ionomycin (Sigma) at 37° C. for 24 h. The ELISpot assay was carried out using a commercial kit (U-CyTech biosciences), following the manufacturer's protocol. The number of human IFN-γ producing cells was expressed as spot-forming units (SFU) per million cells.

Gene Expression Profiling by Quantitative Real-Time PCR

RNA extractions were performed using the RNeasy Mini kit (Qiagen, Valencia, Calif.) and cDNA synthesis was performed with RT kit (Qiagen, Valencia, Calif.), using 1 μg of total RNA. Quantitative RT-PCR (qRT-PCR) was performed on Applied Biosystems 7500 real-time PCR system (Applied Biosystems, Foster City, Calif.) using SYBR-green reagent (Biorad) and custom-made primers (IDT). The sequences of human and mouse mRNAs were extracted from the National Center for Biotechnology Information database. Analogous genes from human and mouse were aligned using the MegAlign software (DNASTAR Lasergene 9 Core Suite) and the species-specific regions for human or mouse were identified for the design of human- or mouse-specific primers, respectively. A complete list of the oligonucleotide primer sequences used is provided in Table 2 below. The relative gene expression values were normalized to species-specific glyceraldehyde-3-phosphate dehydrogenase (GAPDH) and derived using the 2-ΔΔCt method.

TABLE 2
List of primers for quantitative real-time PCR
Quantitative RT-PCR primers

| | Forward primer | Reverse primer |
|---|---|---|
| Gene | (5′-3′) | (5′-3′) |
| Human GAPDH | GCTTAACTCTGGTAAAGT | ATGGAATTTGCCATGG |
| | GGATAT | GTGGAAT |
| Mouse GAPDH | CATTTGCAGTGGCAAAGT | GTTGAATTTGCCGTGA |
| | GGAGA | GTGGAGT |
| Human Col1A1 | CCAATCACCTGCGTACAG | CGTCACAGATCACGTC |
| | AACG | ATCGCA |
| Mouse Co11A1 | AGCTTTGTGGACCTCCGG | CTGACTTCAGGGATGT |
| | CTC | CTTCTTG |
| Human TIMP1 | CCCACAACCGCAGCGAGG | GGCAGGCAAGGTGACG |
| | AG | GGAC |
| Mouse TIMP1 | CTGGCATCTGGCATCCTC | AGTTGCAGAAGGCTGT |
| | TTGT | CTGTGG |

Statistical Analysis

Unpaired two-tailed Student t test was used for all comparisons. P<0.05 is considered as significant. All data are reported as means±standard error of mean.

1. A mouse for human hepatitis studies wherein the mouse has been injected with CD34+ stem cells and wherein the mouse is immunocompromised.

2. The mouse of claim 1, wherein the immunocompromised mouse is characterized by absence of mature T- or B-cells and lack of functional NK cells.

3. The mouse of claim 2, wherein the immunocompromised mouse is further characterized by a deficiency in cytokine signaling.

4. The mouse of any one of claims 2 to 3, wherein the immunocompromised mouse is further characterized by absence of production of detectable serum immunoglobulin

5. The mouse of any one of the preceding claims, wherein the Prkdc-gene of the mouse comprises a severe combined immunodeficiency (scid) mutation (Prkdc^{scid}).

6. The mouse of any one of the preceding claims, wherein the mouse is a NOD scid IL2 receptor gamma chain knockout mouse (NSG) (NOD-SCID-IL2Rγ−/−).

7. The mouse of any one of claims 1 to 6, wherein the CD34+ stem cells are obtained from human fetal tissue.

8. The mouse of any one of claims 1 to 7, wherein the CD34+ stem cells are obtained from human fetal liver.

9. The mouse of claim 8, wherein the CD34+ stem cells used for injection are obtained from freshly harvested human fetal liver.

10. The mouse of claim 8 or 9, wherein the CD34+ stem cells are not obtained from frozen human fetal liver.

11. The mouse of claim 9 or 10, wherein freshly harvested human fetal liver is between about 15 minutes to 2 hours old.

12. The mouse model of any one of claims 1 to 11, wherein the CD34+ stem cells comprise progenitors of the human hepatocytes.

13. The mouse of any one of claims 7 to 12, wherein the CD34+ stem cells essentially consist of CD133(hi) hematopoietic stem cells or consist of CD133(lo) hepatic stem cells.

14. The mouse of any one of the preceding claims, wherein the CD34+ stem cells have been injected by one-step injection.

15. The mouse of any one of claims 7 to 14, wherein the entire population of CD34+ stem cells isolated from human fetal liver is delivered into the mouse.

16. A mouse for human hepatitis studies obtained by a process of injecting CD34+ stem cells into an immunocompromised mouse.

17. The mouse of claim 16, wherein the mouse is characterized as defined in any one of claims 2 to 6.

18. The mouse of claim 16 or 17, wherein the CD34+ stem cells are as defined in any one of claims 7 to 15.

19. The mouse of any one of claims 16 to 18, wherein the CD34+ stem cells have been injected by one-step injection.

20. The mouse of any one of claims 16 to 19, wherein the injection is an intra-hepatic or intra-cardiac injection.

21. The mouse of any one of claims 16 to 20, wherein the process further comprises injecting hepatitis B virus (HBV) or hepatitis C virus (HCV) into the mouse.

22. The mouse of any one of claims 16 to 21, wherein the CD34+ stem cells are injected into a 1 to 3 day old mouse.

23. The mouse of any one of claims 16 to 22, wherein the mouse is capable of developing at least one selected from the group consisting of human leukocyte inflammation, human immune responses and liver damage.

24. The mouse of any one of claims 16 to 22, wherein the mouse is capable of developing human hepatocellular adenoma and human hepatocellular carcinoma after long term HCV infection.

25. The mouse of any one of claims 16 to 24, wherein the mouse is not genetically modified to allow HCV infection.

26. A method of manufacturing a mouse model comprising administering CD34+ stem cells as defined in claims 7 to 15 into an immunocompromised mouse as defined in claims 1 to 25.

27. The method of claim 26, wherein the method further comprises administering HBV or HCV into the mouse.

28. A method of drug screening wherein the method comprises administering anti-HBV or anti-HCV therapeutics to a mouse as defined in any one of claims 1 to 25.

29. A method of characterizing changes in viral quasispecies during HBV or HCV infection by using a mouse as defined in any one of claims 1 to 25.

30. Use of a mouse as defined in any one of claims 1 to 25 for testing the efficiency of putative anti-HBV or anti-HCV drugs or for characterizing changes in viral quasispecies during HBV or HCV infection.
